SEQUENCE LISTING (54 sequences)

SEQ ID NO: 1 - protein, 10 aa, synthetic construct
  REGION 1..10: note = HCDR1
GFTFSSYGMH

SEQ ID NO: 2 - protein, 17 aa, synthetic construct
  REGION 1..17: note = HCDR2
IIWYDGSNKNYADSVKG

SEQ ID NO: 3 - protein, 11 aa, synthetic construct
  REGION 1..11: note = HCDR3
DVNWGNWYFDL

SEQ ID NO: 4 - protein, 11 aa, synthetic construct
  REGION 1..11: note = LCDR1
RASQSVSSNLA

SEQ ID NO: 5 - protein, 7 aa, synthetic construct
  REGION 1..7: note = LCDR2
GASTRAT

SEQ ID NO: 6 - protein, 8 aa, synthetic construct
  REGION 1..8: note = LCDR3
QQYNTWIT

SEQ ID NO: 7 - protein, 120 aa, synthetic construct
  REGION 1..120: note = VH
QVQLVESGGGVVQPGRSLRLSCAASGFTFSSYGMHWVRQAPGKGLEWVAIIWYDGSNKNYADSVKGRFTISRDNSKNTLYLQMNSLRAEDTAVYYCARDVNWGNWYFDLWGRGTLVTVSS

SEQ ID NO: 8 - protein, 106 aa, synthetic construct
  REGION 1..106: note = VL
EVVMTQSPATLSVSPGERATLSCRASQSVSSNLAWYQLKPGQAPRLLIYGASTRATGIPARFSGSGSGTEFTLTISSLQSEDFALYYCQQYNTWITFGQGTRLEIK

SEQ ID NO: 9 - protein, 450 aa, synthetic construct
  REGION 1..450: note = HC
QVQLVESGGGVVQPGRSLRLSCAASGFTFSSYGMHWVRQAPGKGLEWVAIIWYDGSNKNYADSVKGRFTISRDNSKNTLYLQMNSLRAEDTAVYYCARDVNWGNWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, synthetic construct
  REGION 1..213: note = LC
EVVMTQSPATLSVSPGERATLSCRASQSVSSNLAWYQLKPGQAPRLLIYGASTRATGIPARFSGSGSGTEFTLTISSLQSEDFALYYCQQYNTWITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 10 aa, synthetic construct
  REGION 1..10: note = HCDR1
DFTFSSYAMS

SEQ ID NO: 12 - protein, 17 aa, synthetic construct
  REGION 1..17: note = HCDR2
AISGSGGSTYYADSVKG

SEQ ID NO: 13 - protein, 14 aa, synthetic construct
  REGION 1..14: note = HCDR3
GIAVAGLYYYGMDV

SEQ ID NO: 14 - protein, 11 aa, synthetic construct
  REGION 1..11: note = LCDR1
RASQNVISNLA

SEQ ID NO: 15 - protein, 7 aa, synthetic construct
  REGION 1..7: note = LCDR2
TTSTRAT

SEQ ID NO: 16 - protein, 9 aa, synthetic construct
  REGION 1..9: note = LCDR3
QQYNNWPLT

SEQ ID NO: 17 - protein, 123 aa, synthetic construct
  REGION 1..123: note = VH
EVQLLESGGGLVQPGGSLRLSCAASDFTFSSYAMSWVRQAPGKGLEWVSAISGSGGSTYYADSVKGRFTISRDNSKNTLYLQMNSLRAEDTAVYYCAKGIAVAGLYYYGMDVWGQGTTVTVSS

SEQ ID NO: 18 - protein, 107 aa, synthetic construct
  REGION 1..107: note = VL
EIVMTQSPATLSVSPGERATLSCRASQNVISNLAWHQQKPGQAPRLLIYTTSTRATGIPARFSGSGSGTEFTLTISSLQSEDFAVYYCQQYNNWPLTFGGGTKVEIK

SEQ ID NO: 19 - protein, 453 aa, synthetic construct
  REGION 1..453: note = HC
EVQLLESGGGLVQPGGSLRLSCAASDFTFSSYAMSWVRQAPGKGLEWVSAISGSGGSTYYADSVKGRFTISRDNSKNTLYLQMNSLRAEDTAVYYCAKGIAVAGLYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
  REGION 1..214: note = LC
EIVMTQSPATLSVSPGERATLSCRASQNVISNLAWHQQKPGQAPRLLIYTTSTRATGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 10 aa, synthetic construct
  REGION 1..10: note = HCDR1
GFTFSSYAMS

SEQ ID NO: 22 - protein, 17 aa, synthetic construct
  REGION 1..17: note = HCDR2
AISGTTYNTYYAGSVKG

SEQ ID NO: 23 - protein, 14 aa, synthetic construct
  REGION 1..14: note = HCDR3
GISVAGYYYYGMDV

SEQ ID NO: 24 - protein, 11 aa, synthetic construct
  REGION 1..11: note = LCDR1
RASQSVISNLA

SEQ ID NO: 25 - protein, 9 aa, synthetic construct
  REGION 1..9: note = LCDR3
HQYNNWPLT

SEQ ID NO: 26 - protein, 123 aa, synthetic construct
  REGION 1..123: note = VH
EVQLLESGGGLVQPGGSLRLSCAASGFTFSSYAMSWVRQAPGKGLEWVSAISGTTYNTYYAGSVKGRFTISRDNSKNTLYLQMNSLRAEDTAVYYCAKGISVAGYYYYGMDVWGQGTTVTVSS

SEQ ID NO: 27 - protein, 107 aa, synthetic construct
  REGION 1..107: note = VL
EIVMTQSPATLSVSPGERATLSCRASQSVISNLAWYQQKPGQAPRLLIYGASTRATGIPARFSGSGSGTEFTLTISSLQSEDFAVYYCHQYNNWPLTFGGGTKVEIK

SEQ ID NO: 28 - protein, 453 aa, synthetic construct
  REGION 1..453: note = HC
EVQLLESGGGLVQPGGSLRLSCAASGFTFSSYAMSWVRQAPGKGLEWVSAISGTTYNTYYAGSVKGRFTISRDNSKNTLYLQMNSLRAEDTAVYYCAKGISVAGYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 214 aa, synthetic construct
  REGION 1..214: note = LC
EIVMTQSPATLSVSPGERATLSCRASQSVISNLAWYQQKPGQAPRLLIYGASTRATGIPARFSGSGSGTEFTLTISSLQSEDFAVYYCHQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 17 aa, synthetic construct
  REGION 1..17: note = HCDR2
IIWYDGSNRDYADSVKG

SEQ ID NO: 31 - protein, 120 aa, synthetic construct
  REGION 1..120: note = VH
QAQLVESGGGVVQPGRSLRLSCAASGFTFSSYGMHWVRQAPGKGLEWVAIIWYDGSNRDYADSVKGRFTISRDNSKNTLYLQMNSLRAEDTAVYYCARDVNWGNWYFDLWGRGTLVTVSS

SEQ ID NO: 32 - protein, 106 aa, synthetic construct
  REGION 1..106: note = VL
EIVMTQSPATLSVSPGERATLSCRASQSVSSNLAWYQLKPGQAPRLLIYGASTRATGVPARVSGSGSGTEFTLTISSLQSEDFAAYYCQQYNTWITFGQGTRLEIK

SEQ ID NO: 33 - protein, 450 aa, synthetic construct
  REGION 1..450: note = HC
QAQLVESGGGVVQPGRSLRLSCAASGFTFSSYGMHWVRQAPGKGLEWVAIIWYDGSNRDYADSVKGRFTISRDNSKNTLYLQMNSLRAEDTAVYYCARDVNWGNWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 213 aa, synthetic construct
  REGION 1..213: note = LC
EIVMTQSPATLSVSPGERATLSCRASQSVSSNLAWYQLKPGQAPRLLIYGASTRATGVPARVSGSGSGTEFTLTISSLQSEDFAAYYCQQYNTWITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 10 aa, synthetic construct
  REGION 1..10: note = HCDR1
GFVFSSYDMS

SEQ ID NO: 36 - protein, 20 aa, synthetic construct
  REGION 1..20: note = HCDR2
WVAYISSGGGITYAPSTVKG

SEQ ID NO: 37 - protein, 11 aa, synthetic construct
  REGION 1..11: note = HCDR3
HYFGSSGPFAY

SEQ ID NO: 38 - protein, 11 aa, synthetic construct
  REGION 1..11: note = LCDR1
RASENIFSYLA

SEQ ID NO: 39 - protein, 7 aa, synthetic construct
  REGION 1..7: note = LCDR2
NTRTLAE

SEQ ID NO: 40 - protein, 9 aa, synthetic construct
  REGION 1..9: note = LCDR3
QHHYGTPFT

SEQ ID NO: 41 - protein, 120 aa, synthetic construct [sequence omitted]

SEQ ID NO: 42 - protein, 107 aa, synthetic construct [sequence omitted]

SEQ ID NO: 43 - protein, 449 aa, synthetic construct
  REGION 1..449: note = HC
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 214 aa, synthetic construct
  REGION 1..214: note = LC
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 12 aa, synthetic construct
  REGION 1..12: note = HCDR1
GFSLSTYGIGVG

SEQ ID NO: 46 - protein, 16 aa, synthetic construct
  REGION 1..16: note = HCDR2
HIWWNDNKYYSTSLKT

SEQ ID NO: 47 - protein, 8 aa, synthetic construct
  REGION 1..8: note = HCDR3
ISLPYFDY

SEQ ID NO: 48 - protein, 11 aa, synthetic construct
  REGION 1..11: note = LCDR1
KASQNVGTAVA

SEQ ID NO: 49 - protein, 7 aa, synthetic construct
  REGION 1..7: note = LCDR2
SASNRYT

SEQ ID NO: 50 - protein, 9 aa, synthetic construct
  REGION 1..9: note = LCDR3
QQYSSYPLT

SEQ ID NO: 51 - protein, 118 aa, synthetic construct
  REGION 1..118: note = VH
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSS

SEQ ID NO: 52 - protein, 107 aa, synthetic construct
  REGION 1..107: note = VL
DIQLTQSPSFLSASVGDRVTITCKASQNVGTAVAWYQQKPGKAPKLLIYSASNRYTGVPSRFSGSGSGTEFTLTISSLQPEDFATYYCQQYSSYPLTFGGGTKVEIK

SEQ ID NO: 53 - protein, 443 aa, synthetic construct
  REGION 1..443: note = HC
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 54 - protein, 214 aa, synthetic construct
  REGION 1..214: note = LC
DIQLTQSPSFLSASVGDRVTITCKASQNVGTAVAWYQQKPGKAPKLLIYSASNRYTGVPSRFSGSGSGTEFTLTISSLQPEDFATYYCQQYSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0258125 A1
(43) Pub. Date: Sep. 3, 2026

(54) ANTI-CEACAM5/6 DUAL-BINDING ANTIBODIES

(71) Applicant: NONA BIOSCIENCES (SUZHOU) CO., LTD, Suzhou, Jiangsu (CN)

(72) Inventors: Jun WU, Suzhou, Jiangsu (CN); Xiaoxiao WANG, Suzhou, Jiangsu (CN); Xingqun GONG, Suzhou, Jiangsu (CN); Zilai XIANG, Suzhou, Jiangsu (CN); Di WANG, Suzhou, Jiangsu (CN); Zhao KANG, Suzhou, Jiangsu (CN); Lei WU, Suzhou, Jiangsu (CN); Jiayi XU, Suzhou, Jiangsu (CN); Jianhao HOU, Suzhou, Jiangsu (CN); Bowen ZHANG, Suzhou, Jiangsu (CN); Lile LIU, Suzhou, Jiangsu (CN)

(21) Appl. No.: 19/489,959

(22) PCT Filed: May 15, 2024
(86) PCT No.: PCT/CN2024/093329

§ 371 Date: Dec. 4, 2025

(30) Foreign Application Priority Data
Jun. 5, 2023 (WO) PCT/CN2023/098325

(51) Int. Cl. C07K 16/28; A61K 47/68; A61K 49/00; A61K 51/10; A61P 35/00

(52) U.S. Cl. CPC C07K 16/2803; A61K 47/68031; A61K 47/68035; A61K 47/6809; A61K 47/6889; A61K 49/0058; A61K 51/1048; A61P 35/00; C07K 2317/31; C07K 2317/35

(57) ABSTRACT

Antibodies that bind to both CEACAM5 and CEACAM6 and antigen-binding fragments thereof, as well as uses thereof, nucleic acids encoding the antibodies and antigen-binding fragments, vectors comprising the nucleic acids, and host cell comprising the nucleic acids or the vectors. The pharmaceutical compositions and conjugates comprising the antibodies, and therapeutic methods by administering the antibodies.

### FIELD OF THE INVENTION

The invention relates to antibodies and antigen-binding fragments thereof that bind to both CEACAM5 and CEACAM6, and uses thereof.

### BACKGROUND OF THE INVENTION

Carcino-embryonic antigen (CEA) is a glycoprotein involved in cell adhesion. CEA was first identified in 1965 (Gold and Freedman, J Exp Med, 121, 439, 1965) as a protein normally expressed by fetal gut during the first six months of gestation, and found in cancers of the pancreas, liver and colon. The CEA family belongs to the immunoglobulin superfamily. The CEA family, which consists of 18 genes, is subdivided in two sub-groups of proteins: the carcinoembryonic antigen-related cell adhesion molecule (CEACAM) sub-group and the pregnancy-specific glycoprotein subgroup (Kammerer & Zimmermann, BMC Biology 2010, 8:12). In humans, the CEACAM sub-group consists of 7 members: CEACAM1, CEACAM3, CEACAM4, CEACAM5, CEACAM6, CEACAM7, CEACAM8.

CEACAM5 (Carcinoembryonic antigen-related cell adhesion molecule 5), also known as CEA or CD66e, is a well-known tumor associated antigen, which has been used as a biomarker in cancer diagnostics for years. Expression of CEACAM5 is highly correlated with certain cancers. It is upregulated in colon, rectum, lung, pancreas and stomach cancers, with up to several hundreds fold change comparing to normal tissue. The distribution profile of CEACAM5 makes it a very good target for therapeutics such as antibody and ADC (antibody drug conjugate). For example, Sanofi's SAR408701 (tusamitamab ravtansine) comprises the anti-CEACAM5 antibody SAR408377 (tusamitamab; also referred to as huMab2-3) covalently linked to the cytotoxic agent DM4, a potent microtubule-destabilizing maytansinoid, via an N-succinimidyl 4-(2-pyridyldithio) butyrate (SPDB) linker.

CEACAM6 (Carcinoembryonic antigen-related cell adhesion molecule 6), also known as NCA90 or CD66c, is also a well-known tumor associated antigen and has been used as a biomarker in cancer diagnostics. Expression of CEACAM6 is also highly correlated with certain cancers, such as colon, rectum, lung, pancreas and stomach cancers. CEACAM6 is also a very good target for therapeutics such as antibody and ADC due to its distribution profile.

In cancers with CEACAM5 and CEACAM6 co-expressed, one may have even higher expression than the other. Therefore, antibody targeting both CEACAM5 and CEACAM6 may cover more cancer cells in a heterogeneous tumor microenvironment and thus reach larger patient groups. Therefore, it is important in tumor immunotherapy to develop antibodies that target CEACAM5 and CEACAM6 simultaneously.

### SUMMARY OF THE INVENTION

The invention provides antibodies that bind to both CEACAM5 and CEACAM6 and antigen-binding fragments thereof.

In a first aspect, the invention provides an antibody that binds to both CEACAM5 and CEACAM6, or an antigen binding fragment thereof, wherein the antibody comprises a heavy chain variable region (VH) and a light chain variable region (VL), and wherein
(1) the VH comprises the HCDRs 1-3 of a VH having the amino acid sequence set forth in SEQ ID NO: 26, and the VL comprises the LCDRs 1-3 of a VL having the amino acid sequence set forth in SEQ ID NO: 27;
(2) the VH comprises the HCDRs 1-3 of a VH having the amino acid sequence set forth in SEQ ID NO: 7, and the VL comprises the LCDRs 1-3 of a VL having the amino acid sequence set forth in SEQ ID NO: 8;
(3) the VH comprises the HCDRs 1-3 of a VH having the amino acid sequence set forth in SEQ ID NO: 17, and the VL comprises the LCDRs 1-3 of a VL having the amino acid sequence set forth in SEQ ID NO: 18; or
(4) the VH comprises the HCDRs 1-3 of a VH having the amino acid sequence set forth in SEQ ID NO: 31, and the VL comprises the LCDRs 1-3 of a VL having the amino acid sequence set forth in SEQ ID NO: 32.

In some embodiments, the invention provides an antibody that binds to both CEACAM5 and CEACAM6, or an antigen binding fragment thereof, wherein the antibody comprises a heavy chain variable region (VH) and a light chain variable region (VL), and wherein
(1) the VH comprises HCDRs 1-3 comprising the amino acid sequences set forth in SEQ ID NOS: 21, 22 and 23 respectively, and the VL comprises LCDRs 1-3 comprising the amino acid sequences set forth in SEQ ID NOs: 24, 5 and 25 respectively;
(2) the VH comprises HCDRs 1-3 comprising the amino acid sequences set forth in SEQ ID NOs: 1, 2 and 3 respectively, and the VL comprises LCDRs 1-3 comprising the amino acid sequences set forth in SEQ ID NOs: 4, 5 and 6 respectively;
(3) the VH comprises HCDRs 1-3 comprising the amino acid sequences set forth in SEQ ID NOs: 11, 12 and 13 respectively, and the VL comprises LCDRs 1-3 comprising the amino acid sequences set forth in SEQ ID NOs: 14, 15 and 16 respectively; or
(4) the VH comprises HCDRs 1-3 comprising the amino acid sequences set forth in SEQ ID NOs: 1, 30 and 3 respectively, and the VL comprises LCDRs 1-3 comprising the amino acid sequences set forth in SEQ ID NOs: 4, 5 and 6 respectively.

In some embodiments, the invention provides an antibody that binds to both CEACAM5 and CEACAM6, or an antigen binding fragment thereof, wherein the antibody comprises a heavy chain variable region (VH) and a light chain variable region (VL), and wherein
(1) the VH comprises an amino acid sequence having at least 80%, at least 85%, at least 90%, at least 95%, at least 98%, at least 99%, or 100% sequence identity to SEQ ID NO: 26, and the VL comprises an amino acid sequence having at least 80%, at least 85%, at least 90%, at least 95%, at least 98%, at least 99%, or 100% sequence identity to SEQ ID NO: 27;
(2) the VH comprises an amino acid sequence having at least 80%, at least 85%, at least 90%, at least 95%, at least 98%, at least 99%, or 100% sequence identity to SEQ ID NO: 7, and the VL comprises an amino acid sequence having at least 80%, at least 85%, at least 90%, at least 95%, at least 98%, at least 99%, or 100% sequence identity to SEQ ID NO: 8;
(3) the VH comprises an amino acid sequence having at least 80%, at least 85%, at least 90%, at least 95%, at least 98%, at least 99%, or 100% sequence identity to SEQ ID NO: 17, and the VL comprises an amino acid sequence having at least 80%, at least 85%, at least 90%, at least 95%, at least 98%, at least 99%, or 100% sequence identity to SEQ ID NO: 18; or
(4) the VH comprises an amino acid sequence having at least 80%, at least 85%, at least 90%, at least 95%, at least 98%, at least 99%, or 100% sequence identity to SEQ ID NO: 31, and the VL comprises an amino acid sequence having at least 80%, at least 85%, at least 90%, at least 95%, at least 98%, at least 99%, or 100% sequence identity to SEQ ID NO: 32.

In preferred embodiments, the invention provides an antibody that binds to both CEACAM5 and CEACAM6, or an antigen binding fragment thereof, wherein the antibody comprises a heavy chain variable region (VH) and a light chain variable region (VL), and wherein
(1) the VH comprises an amino acid sequence set forth in SEQ ID NO: 26, and the VL comprises an amino acid sequence set forth in SEQ ID NO: 27;
(2) the VH comprises an amino acid sequence set forth in SEQ ID NO: 7, and the VL comprises an amino acid sequence set forth in SEQ ID NO: 8;
(3) the VH comprises an amino acid sequence set forth in SEQ ID NO: 17, and the VL comprises an amino acid sequence set forth in SEQ ID NO: 18; or
(4) the VH comprises an amino acid sequence set forth in SEQ ID NO: 31, and the VL comprises an amino acid sequence set forth in SEQ ID NO: 32.

In some embodiments, the antibody comprises a heavy chain (HC) and a light chain (LC), wherein:
(1) the HC comprises an amino acid sequence having at least 80%, at least 85%, at least 90%, at least 95%, at least 98%, at least 99%, or 100% sequence identity to SEQ ID NO: 28, and the LC comprises an amino acid sequence having at least 80%, at least 85%, at least 90%, at least 95%, at least 98%, at least 99%, or 100% sequence identity to SEQ ID NO: 29;
(2) the HC comprises an amino acid sequence having at least 80%, at least 85%, at least 90%, at least 95%, at least 98%, at least 99%, or 100% sequence identity to SEQ ID NO: 9, and the LC comprises an amino acid sequence having at least 80%, at least 85%, at least 90%, at least 95%, at least 98%, at least 99%, or 100% sequence identity to SEQ ID NO: 10;
(3) the HC comprises an amino acid sequence having at least 80%, at least 85%, at least 90%, at least 95%, at least 98%, at least 99%, or 100% sequence identity to SEQ ID NO: 19, and the LC comprises an amino acid sequence having at least 80%, at least 85%, at least 90%, at least 95%, at least 98%, at least 99%, or 100% sequence identity to SEQ ID NO: 20; or
(4) the HC comprises an amino acid sequence having at least 80%, at least 85%, at least 90%, at least 95%, at least 98%, at least 99%, or 100% sequence identity to SEQ ID NO: 33, and the LC comprises an amino acid sequence having at least 80%, at least 85%, at least 90%, at least 95%, at least 98%, at least 99%, or 100% sequence identity to SEQ ID NO: 34.

In preferred embodiments, the antibody comprises a heavy chain (HC) and a light chain (LC), wherein:
(1) the HC comprises an amino acid sequence set forth in SEQ ID NO: 28, and the LC comprises an amino acid sequence set forth in SEQ ID NO: 29;
(2) the HC comprises an amino acid sequence set forth in SEQ ID NO: 9, and the LC comprises an amino acid sequence set forth in SEQ ID NO: 10;
(3) the HC comprises an amino acid sequence set forth in SEQ ID NO: 19, and the LC comprises an amino acid sequence set forth in SEQ ID NO: 20; or
(4) the HC comprises an amino acid sequence set forth in SEQ ID NO: 33, and the LC comprises an amino acid sequence set forth in SEQ ID NO: 34.

In some embodiments, the antibody is a murine antibody, a chimeric antibody, a humanized antibody, or a human antibody.

In some embodiments, the antibody is of an isotype selected from the group consisting of IgG, IgA, IgM, IgE and IgD.

In some embodiments, the antibody is of a subtype selected from the group consisting of IgG1, IgG2, IgG3, and IgG4.

In some embodiments, the antigen binding fragment is selected from the group consisting of Fab, Fab′, F(ab′)₂, Fd, Fd′, Fv, scFv, ds-scFv and dAb.

In some embodiments, the antibody is a monoclonal antibody, a bi-specific or a multi-specific antibody.

In some embodiments, the antibody is monovalent, bivalent or multivalent.

In some embodiments, the antibody or antigen binding fragment is attached to a fluorescent label, radiolabel or a drug moiety.

In some embodiments, the antibody or antigen binding fragment is attached to a drug moiety, and the drug moiety is selected from the group consisting of microtubule inhibitors, antibiotics, DNA synthesis inhibitors, topoisomerase inhibitors, RNA polymerase II inhibitors, and RNA spliceosome inhibitors.

In preferred embodiments, the drug moiety is selected from the group consisting of MMAE, MMAF, duocarmycin, DM1, DM4, SN-38, Dxd, calicheamicin, doxorubicin, and PBDs (Benzodiazepines).

In some embodiments, the antibody or antigen binding fragment is attached to the drug moiety via a linker.

In some embodiments, the linker comprises a cleavable linker or a non-cleavable linker.

In some embodiments, the cleavable linker is selected from the group consisting of an acid-labile linker, a hydrophilic linker, a protease-sensitive linker, a photolabile linker, a hydrazone linker, a dimethyl linker, and a disulfide-containing linker.

In preferred embodiments, the linker is selected from the group consisting of MC (6-maleimidocaproyl), Val-Cit (valine-citrulline), PABC (para-amino-benzyloxycarbonyl), DMEA (dimethylethylamine), Val-Cit-PABC, MC-Val-Cit-PABC, MC-Val-Cit-PABC-DMEA, GGFG (Glycine-Glycine-Phenylalanine-Glycine), MC-GGFG-aminomethyl, AcBut (4-(4-acetylphenoxy)-butanoic acid), and AcBut-dimethylhydrazide, preferably MC-Val-Cit-PABC.

In a second aspect, the invention provides a bi-specific antibody, comprising the antibody or antigen-binding fragment thereof of the first aspect of the invention and a second antigen binding region specifically binding to a tumor associated antigen, an immune cell antigen, or an immune checkpoint molecule.

In the third aspect, the invention provides a nucleic acid comprising a nucleotide sequence encoding the antibody or the antigen binding fragment thereof of the first aspect of the invention or the bi-specific antibody of the second aspect of the invention.

In the fourth aspect, the invention provides a vector comprising the nucleic acid of the third aspect of the invention.

In the fifth aspect, the invention provides a host cell comprising the nucleic acid of the third aspect of the invention or the vector of the fourth aspect of the invention.

In the sixth aspect, the invention provides an antibody-drug conjugate (ADC), wherein the ADC comprises the antibody or the antigen-binding fragment thereof of the first aspect of the invention or the bi-specific antibody of the second aspect of the invention, and a drug moiety conjugated thereto via a linker.

In some embodiments, the drug moiety is selected from the group consisting of microtubule inhibitors, antibiotics, DNA synthesis inhibitors, topoisomerase inhibitors, RNA polymerase II inhibitors, and RNA spliceosome inhibitors.

In preferred embodiments, the drug moiety is selected from the group consisting of MMAE, MMAF, duocarmycin, DM1, DM4, SN-38, Dxd, calicheamicin, doxorubicin, and PBDs (Benzodiazepines).

In some embodiments, the linker comprises a cleavable linker or a non-cleavable linker.

In some embodiments, the cleavable linker is selected from the group consisting of an acid-labile linker, a hydrophilic linker, a protease-sensitive linker, a photolabile linker, a hydrazone linker, a dimethyl linker, and a disulfide-containing linker.

In preferred embodiments, the linker is selected from the group consisting of MC (6-maleimidocaproyl), Val-Cit (valine-citrulline), PABC (para-amino-benzyloxycarbonyl), DMEA (dimethylethylamine), Val-Cit-PABC, MC-Val-Cit-PABC, MC-Val-Cit-PABC-DMEA, GGFG (Glycine-Glycine-Phenylalanine-Glycine), MC-GGFG-aminomethyl, AcBut (4-(4-acetylphenoxy)-butanoic acid), and AcBut-dimethylhydrazide, preferably MC-Val-Cit-PABC.

In the seventh aspect, the invention provides a pharmaceutical composition comprising (i) the antibody or the antigen binding fragment thereof of the first aspect of the invention, or the bi-specific antibody of the second aspect of the invention, or the nucleic acid of the third aspect of the invention, or the vector of the fourth aspect of the invention, or the host cell of the fifth aspect of the invention, or the antibody-drug conjugate of the sixth aspect of the invention; and optionally (ii) a pharmaceutically acceptable carrier or excipient.

In some embodiments, the composition further comprises a second therapeutic agent. Preferably, the second therapeutic agent is selected from the group consisting of an antibody, a chemotherapeutic agent, an siRNA, an antisense oligonucleotide, a polypeptide, and a small molecule drug.

In the eighth aspect, the invention provides a method of treating a cancer in a subject, comprising administering to the subject an effective amount of the antibody or the antigen binding fragment thereof, the bi-specific antibody, the nucleic acid, the vector, the host cell, the antibody-drug conjugate, or the pharmaceutical composition of the invention.

In some specific embodiments, the cancer is a cancer associated with the expression of CEACAM5 and/or CEACAM6. In preferred embodiments, the cancer is selected from the group consisting of small intestine cancer, colorectal cancer, stomach cancer, lung cancer, cervical cancer, pancreatic cancer, esophagus cancer, ovarian cancer, thyroid cancer, bladder cancer, endometrial cancer, breast cancer, liver cancer, prostatic cancer and skin cancer.

In some embodiments, the method further comprises administering to the subject a second therapeutic agent. Preferably, the second therapeutic agent is selected from an antibody, a chemotherapeutic agent, an siRNA, an antisense oligonucleotide, a polypeptide, and a small molecule drug.

In a ninth aspect, the invention provides use of the antibody or the antigen binding fragment thereof, the bi-specific antibody, the nucleic acid, the vector, the host cell, the antibody-drug conjugate, or the pharmaceutical composition of the invention in the manufacture of a medicament for treating a cancer in a subject.

In some embodiments, the cancer is a cancer associated with the expression of CEACAM5 and/or CEACAM6. In preferred embodiments, the cancer is selected from the group consisting of small intestine cancer, colorectal cancer, stomach cancer, lung cancer, cervical cancer, pancreatic cancer, esophagus cancer, ovarian cancer, thyroid cancer, bladder cancer, endometrial cancer, breast cancer, liver cancer, prostatic cancer and skin cancer.

In some embodiments, the medicament further comprises a second therapeutic agent. Preferably, the second therapeutic agent is selected from an antibody, a chemotherapeutic agent, an siRNA, an antisense oligonucleotide, a polypeptide, and a small molecule drug.

In some embodiments, the medicament is administered in combination with a second therapeutic agent. Preferably, the second therapeutic agent is selected from an antibody, a chemotherapeutic agent, an siRNA, antisense oligonucleotide, a polypeptide, and a small molecule drug.

In a tenth aspect, the invention provides the antibody or the antigen binding fragment thereof, the bi-specific antibody, the nucleic acid, the vector, the host cell, the antibody-drug conjugate, or the pharmaceutical composition of the invention for use in a method of treating a cancer in a subject.

In some embodiments, the cancer is a cancer associated with the expression of CEACAM5 and/or CEACAM6. In preferred embodiments, the cancer is selected from the group consisting of small intestine cancer, colorectal cancer, stomach cancer, lung cancer, cervical cancer, pancreatic cancer, esophagus cancer, ovarian cancer, thyroid cancer, bladder cancer, endometrial cancer, breast cancer, liver cancer, prostatic cancer and skin cancer.

In some embodiments, a second therapeutic agent is further administered the subject. Preferably, the second therapeutic agent is selected from an antibody, a chemotherapeutic agent, an siRNA, antisense oligonucleotide, a polypeptide, and a small molecule drug.

In an eleventh aspect, the invention provides a method for detecting the presence or level of CEACAM5 and/or CEACAM6 in a sample, comprising:
(a) contacting the sample with the antibody or the antigen binding fragment thereof according to any one of claims 1-10, and
(b) determining the presence or level of CEACAM5 and/or CEACAM6 in the sample by detecting binding of the antibody to the sample.

In a preferred embodiment, the method is used for a non-diagnostic purpose.

In a twelfth aspect, the invention provides a method for diagnosing a cancer associated with the expression of CEACAM5 and/or CEACAM6 in a subject comprising:
(a) obtaining a biological sample from the subject,
(b) contacting the sample with the antibody or the antigen binding fragment thereof of the invention, and
(c) detecting binding of the antibody to the sample, wherein an increase in binding of the antibody or antigen binding fragment thereof to the sample as compared to binding of the antibody or antigen binding fragment thereof to a control sample identifies the subject as having the cancer.

In a thirteenth aspect, the invention provides a method for imaging a cancer associated with the expression of CEACAM5 and/or CEACAM6 in a subject comprising:
(a) administering the antibody or antigen binding fragment thereof of the invention to the subject, wherein the antibody is conjugated to a detectable marker, and
(b) detecting the presence of the marker.

In some embodiments, the detectable marker is 111 In, and preferably the detection of the marker is by single-photon emission computed tomography. In other embodiments, the detectable marker is 89Zr, and preferably the detection of the marker is by positron emission tomography.

### DESCRIPTION OF THE DRAWINGS

FIG. 1 illustrates the procedure of immunization and single B cell cloning to generate anti-CEACAM5/6 antibodies.

FIG. 2 illustrates the binding of anti-CEACAM5/6 dual-binding antibodies to human CEACAM5 protein.

FIG. 3 illustrates the binding of anti-CEACAM5/6 dual-binding antibodies to cyno CEACAM5 protein.

FIG. 4 illustrates the binding of anti-CEACAM5/6 dual-binding antibodies to human CEACAM6 protein.

FIG. 5 illustrates no binding of anti-CEACAM5/6 dual-binding antibodies to human CEACAM1 protein.

FIG. 6 illustrates no binding of anti-CEACAM5/6 dual-binding antibodies to human CEACAM3 protein.

FIG. 7 illustrates no binding of anti-CEACAM5/6 dual-binding antibodies to human CEACAM8 protein.

FIG. 8 illustrates the binding of anti-CEACAM5/6 dual-binding antibodies to 293T cells overexpressing human CEACAM5.

FIG. 9 illustrates the binding of anti-CEACAM5/6 dual-binding antibodies to 293T cells overexpressing human CEACAM6.

FIG. 10 illustrates the binding of anti-CEACAM5/6 dual-binding antibodies to CHO-K1 cells overexpressing cyno CEACAM5 or cyno CEACAM6.

FIG. 11 illustrates the binding of anti-CEACAM5/6 dual-binding antibodies to cancer cell lines LS 174T, BxPC3 and A549.

FIG. 12 illustrates internalization of anti-CEACAM5/6 dual-binding antibodies on 293T cell lines overexpressing human CEACAM5 or human CEACAM6, and cancer cell lines LS 174T, HPAC (pancreas cancer) and BxPC3.

FIG. 13 illustrates internalization of anti-CEACAM5/6 dual-binding antibodies on cancer cell lines LS 174T, A549 (lung cancer), BxPC3 and HPAC in the presence of 100 nM soluble human CEACAM5 or human CEACAM6 with soluble protein interference.

FIG. 14 illustrates the evaluation of anti-tumor activity of anti-CEACAM5/6 ADCs against MKN45 gastric cancer in CDX mouse model.

FIG. 15 illustrates the evaluation of anti-tumor activity of anti-CEACAM5/6 ADCs against LS 174T colon cancer in CDX mouse model.

### SEQUENCES

The amino acid sequences of CDRs (according to Kabat and Chothia Combined system), heavy chain variable region (VH), light chain variable region (VL), whole heavy chain (HC) and light chain (LC) of the anti-CEACAM5/6 antibodies of the invention PR304169, PR304171, PR304294, and PR304713 are indicated in Table 1. The amino acid sequences of CDRs (according to Kabat and Chothia Combined system), heavy chain variable region (VH), light chain variable region (VL), whole heavy chain (HC) and light chain (LC) of anti-CEACAM5 reference antibody PR303561 (Tusamitamab) and anti-CEACAM6 reference antibody PR303563 (Tinurilimab) are indicated in Table 2.

TABLE 1
Amino acid sequences of the antibodies of the invention

| Description | Sequence | SEQ ID NO |
|---|---|---|
| PR304169 | | |
| HCDR1 | GFTFSSYGMH | 1 |
| HCDR2 | IIWYDGSNKNYADSVKG | 2 |
| HCDR3 | DVNWGNWYFDL | 3 |
| LCDR1 | RASQSVSSNLA | 4 |
| LCDR2 | GASTRAT | 5 |
| LCDR3 | QQYNTWIT | 6 |
| VH | QVQLVESGGGVVQPGRSLRLSCAASGFTFSSYGMHWVRQAPGKGL | 7 |
| | EWVAIIWYDGSNKNYADSVKGRFTISRDNSKNTLYLQMNSLRAED | |
| | TAVYYCARDVNWGNWYFDLWGRGTLVTVSS | |
| VL | EVVMTQSPATLSVSPGERATLSCRASQSVSSNLAWYQLKPGQAPRL | 8 |
| | LIYGASTRATGIPARFSGSGSGTEFTLTISSLQSEDFALYYCQQYNTW | |
| | ITFGQGTRLEIK | |
| HC | QVQLVESGGGVVQPGRSLRLSCAASGFTFSSYGMHWVRQAPGKGL | 9 |
| | EWVAIIWYDGSNKNYADSVKGRFTISRDNSKNTLYLQMNSLRAED | |
| | TAVYYCARDVNWGNWYFDLWGRGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVV | |
| | SVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPP | |
| | VLDSDGSFFLYSKLTVDKSRWQQGNVFSCSVMHEALHNHYTQKSL | |
| | SLSPGK | |
| LC | EVVMTQSPATLSVSPGERATLSCRASQSVSSNLAWYQLKPGQAPRL | 10 |
| | LIYGASTRATGIPARFSGSGSGTEFTLTISSLQSEDFALYYCQQYNTW | |
| | ITFGQGTRLEIK | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |
| PR304171 | | |
| HCDR1 | DFTFSSYAMS | 11 |
| HCDR2 | AISGSGGSTYYADSVKG | 12 |
| HCDR3 | GIAVAGLYYYGMDV | 13 |
| LCDR1 | RASQNVISNLA | 14 |
| LCDR2 | TTSTRAT | 15 |
| LCDR3 | QQYNNWPLT | 16 |
| VH | EVQLLESGGGLVQPGGSLRLSCAASDFTFSSYAMSWVRQAPGKGLE | 17 |
| | WVSAISGSGGSTYYADSVKGRFTISRDNSKNTLYLQMNSLRAEDTA | |
| | VYYCAKGIAVAGLYYYGMDVWGQGTTVTVSS | |
| VL | EIVMTQSPATLSVSPGERATLSCRASQNVISNLAWHQQKPGQAPRLL | 18 |
| | IYTTSTRATGIPARFSGSGSGTEFTLTISSLQSEDFAVYYCQQYNNW | |
| | PLTFGGGTKVEIK | |
| HC | EVQLLESGGGLVQPGGSLRLSCAASDFTFSSYAMSWVRQAPGKGLE | 19 |
| | WVSAISGSGGSTYYADSVKGRFTISRDNSKNTLYLQMNSLRAEDTA | |
| | VYYCAKGIAVAGLYYYGMDVWGQGTTVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVV | |
| | SVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPP | |
| | VLDSDGSFFLYSKLTVDKSRWQQGNVFSCSVMHEALHNHYTQKSL | |
| | SLSPGK | |
| LC | EIVMTQSPATLSVSPGERATLSCRASQNVISNLAWHQQKPGQAPRLL | 20 |
| | IYTTSTRATGIPARFSGSGSGTEFTLTISSLQSEDFAVYYCQQYNNW | |
| | PLTFGGGTKVEIK | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |
| PR304294 | | |
| HCDR1 | GFTFSSYAMS | 21 |
| HCDR2 | AISGTTYNTYYAGSVKG | 22 |
| HCDR3 | GISVAGYYYYGMDV | 23 |
| LCDR1 | RASQSVISNLA | 24 |
| LCDR2 | GASTRAT | 5 |
| LCDR3 | HQYNNWPLT | 25 |
| VH | EVQLLESGGGLVQPGGSLRLSCAASGFTFSSYAMSWVRQAPGKGLE | 26 |
| | WVSAISGTTYNTYYAGSVKGRFTISRDNSKNTLYLQMNSLRAEDTA | |
| | VYYCAKGISVAGYYYYGMDVWGQGTTVTVSS | |
| VL | EIVMTQSPATLSVSPGERATLSCRASQSVISNLAWYQQKPGQAPRLL | 27 |
| | IYGASTRATGIPARFSGSGSGTEFTLTISSLQSEDFAVYYCHQYNNWPLT | |
| | FGGGTKVEIK | |
| HC | EVQLLESGGGLVQPGGSLRLSCAASGFTFSSYAMSWVRQAPGKGLE | 28 |
| | WVSAISGTTYNTYYAGSVKGRFTISRDNSKNTLYLQMNSLRAEDTA | |
| | VYYCAKGISVAGYYYYGMDVWGQGTTVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVV | |
| | SVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPP | |
| | VLDSDGSFFLYSKLTVDKSRWQQGNVFSCSVMHEALHNHYTQKSL | |
| | SLSPGK | |
| LC | EIVMTQSPATLSVSPGERATLSCRASQSVISNLAWYQQKPGQAPRLL | 29 |
| | IYGASTRATGIPARFSGSGSGTEFTLTISSLQSEDFAVYYCHQYNNWP | |
| | LTFGGGTKVEIK | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |
| PR304713 | | |
| HCDR1 | GFTFSSYGMH | 1 |
| HCDR2 | IIWYDGSNRDYADSVKG | 30 |
| HCDR3 | DVNWGNWYFDL | 3 |
| LCDR1 | RASQSVSSNLA | 4 |
| LCDR2 | GASTRAT | 5 |
| LCDR3 | QQYNTWIT | 6 |
| VH | QAQLVESGGGVVQPGRSLRLSCAASGFTFSSYGMHWVRQAPGKGL | 31 |
| | EWVAIIWYDGSNRDYADSVKGRFTISRDNSKNTLYLQMNSLRAEDT | |
| | AVYYCARDVNWGNWYFDLWGRGTLVTVSS | |
| VL | EIVMTQSPATLSVSPGERATLSCRASQSVSSNLAWYQLKPGQAPRLL | 32 |
| | IYGASTRATGVPARVSGSGSGTEFTLTISSLQSEDFAAYYCQQYNTW | |
| | ITFGQGTRLEIK | |
| HC | QAQLVESGGGVVQPGRSLRLSCAASGFTFSSYGMHWVRQAPGKGL | 33 |
| | EWVAIIWYDGSNRDYADSVKGRFTISRDNSKNTLYLQMNSLRAEDT | |
| | AVYYCARDVNWGNWYFDLWGRGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVV | |
| | SVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPP | |
| | VLDSDGSFFLYSKLTVDKSRWQQGNVFSCSVMHEALHNHYTQKSL | |
| | SLSPGK | |
| LC | EIVMTQSPATLSVSPGERATLSCRASQSVSSNLAWYQLKPGQAPRLL | 34 |
| | IYGASTRATGVPARVSGSGSGTEFTLTISSLQSEDFAAYYCQQYNTW | |
| | ITFGQGTRLEIK | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |

TABLE 2
Amino acid sequences of the reference antibodies

| Description | Sequence | SEQ ID NO |
|---|---|---|
| PR303561 | | |
| HCDR1 | GFVFSSYDMS | 35 |
| HCDR2 | WVAYISSGGGITYAPSTVKG | 36 |
| HCDR3 | HYFGSSGPFAY | 37 |
| LCDR1 | RASENIFSYLA | 38 |
| LCDR2 | NTRTLAE | 39 |
| LCDR3 | QHHYGTPFT | 40 |
| VH | EVQLQESGPGLVKPGGSLSLSCAASGFVFSSYDMSWVRQTPERGLE | 41 |
| | WVAYISSGGGITYAPSTVKGRFTVSRDNAKNTLYLQMNSLTSEDTA | |
| | VYYCAAHYFGSSGPFAYWGQGTLVTVSS | |
| VL | DIQMTQSPASLSASVGDRVTITCRASENIFSYLAWYQQKPGKSPKLL | 42 |
| | VYNTRTLAEGVPSRFSGSGSGTDFSLTISSLQPEDFATYYCQHHYGT | |
| | PFTFGSGTKLEIK | |
| HC | EVQLQESGPGLVKPGGSLSLSCAASGFVFSSYDMSWVRQTPERGLE | 43 |
| | WVAYISSGGGITYAPSTVKGRFTVSRDNAKNTLYLQMNSLTSEDTA | |
| | VYYCAAHYFGSSGPFAYWGQGTLVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVV | |
| | SVLTVLHQDWLNGKEYKCKVSNKALPAPIEKTISKAKGQPREPQVY | |
| | TLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSL | |
| | SLSPG | |
| LC | DIQMTQSPASLSASVGDRVTITCRASENIFSYLAWYQQKPGKSPKLL | 44 |
| | VYNTRTLAEGVPSRFSGSGSGTDFSLTISSLQPEDFATYYCQHHYGT | |
| | PFTFGSGTKLEIK | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |
| PR303563 | | |
| HCDR1 | GFSLSTYGIGVG | 45 |
| HCDR2 | HIWWNDNKYYSTSLKT | 46 |
| HCDR3 | ISLPYFDY | 47 |
| LCDR1 | KASQNVGTAVA | 48 |
| LCDR2 | SASNRYT | 49 |
| LCDR3 | QQYSSYPLT | 50 |
| VH | QVTLRESGPALVKPTQTLTLTCTFSGFSLSTYGIGVGWIRQPPGKAL | 51 |
| | EWLAHIWWNDNKYYSTSLKTRLTISKDTSKNQVVLTMTNMDPVDT | |
| | ATYYCARISLPYFDYWGQGTTLTVSS | |
| VL | DIQLTQSPSFLSASVGDRVTITCKASQNVGTAVAWYQQKPGKAPKL | 52 |
| | LIYSASNRYTGVPSRFSGSGSGTEFTLTISSLQPEDFATYYCQQYS | |
| | SYPLTFGGGTKVEIK | |
| HC | QVTLRESGPALVKPTQTLTLTCTFSGFSLSTYGIGVGWIRQPPGKAL | 53 |
| | EWLAHIWWNDNKYYSTSLKTRLTISKDTSKNQVVLTMTNMDPVDT | |
| | ATYYCARISLPYFDYWGQGTTLTVSS | |
| | ASTKGPSVFPLAPCSRSTSESTAALGCLVKDYFPEPVTVSWNSGALT | |
| | SGVHTFPAVLQSSGLYSLSSVVTVPSSNFGTQTYTCNVDHKPSNTKV | |
| | DKTVERKCCVECPPCPAPPVAGPSVFLFPPKPKDTLMISRTPEVTCV | |
| | VVDVSHEDPEVQFNWYVDGVEVHNAKTKPREEQFNSTFRVVSVLT | |
| | VVHQDWLNGKEYKCKVSNKGLPAPIEKTISKTKGQPREPQVYTLPP | |
| | SREEMTKNQVSLTCLVKGFYPSDIAVEWESNGQPENNYKTTPPMLD | |
| | SDGSFFLYSKLTVDKSRWQQGNVFSCSVMHEALHNHYTQKSLSLSP | |
| | G | |
| LC | DIQLTQSPSFLSASVGDRVTITCKASQNVGTAVAWYQQKPGKAPKL | 54 |
| | LIYSASNRYTGVPSRFSGSGSGTEFTLTISSLOPEDFATYYCQQYSSY | |
| | PLTFGGGTKVEIK | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |

### DETAILED DESCRIPTION OF THE INVENTION

The aforementioned features and advantages of the invention as well as additional features and advantages thereof will be more clearly understood hereafter as a result of a detailed description of the following embodiments when taken in conjunction with the drawings.

The embodiments described herein with reference to drawings are explanatory, illustrative, and used to generally understand the present invention. The embodiments shall not be construed to limit the scope of the present invention. The same or similar elements and the elements having same or similar functions are denoted by like reference numerals throughout the descriptions. Unless indicated or defined otherwise, all terms used have their usual meaning in the art, which will be clear to the skilled person. Reference is for example made to the standard handbooks, such as Leuenberger, H. G. W, Nagel, B. and Klbl, H. eds., “A multilingual glossary of biotechnological terms: (IUPAC Recommendations)”, Helvetica Chimica Acta (1995), CH-4010 Basel, Switzerland; Sambrook et al, “Molecular Cloning: A Laboratory Manual” (2nd Ed.), Vols. 1-3, Cold Spring Harbor Laboratory Press (1989); F. Ausubel et al, eds., “Current protocols in molecular biology”, Green Publishing and Wiley InterScience, New York (1987); Roitt et al., “Immunology (6th Ed.), Mosby/Elsevier, Edinburgh (2001); and Janeway et al., “Immunobiology” (6th Ed.), Garland Science Publishing/Churchill Livingstone, New York (2005), as well as the general background art cited above.

As used herein, singular forms “a”, “an” and “the” include plural referents unless the context clearly indicates otherwise. Thus, for example, reference to “an antibody” includes a plurality of antibodies.

Unless indicated or defined otherwise, the term “comprise”, and variations thereof such as “comprises” and “comprising”, should be understood to imply the inclusion of a stated elements or step or a group of elements or steps but not the exclusion of any other element or step or a group of elements or steps. The term “comprising” encompasses “including” as well as “consisting” e.g., a composition “comprising” X may consist exclusively of X or may include something additional e.g., X+Y.

The term “about” in relation to a numerical value x is optional and means, for example, x±10% or x±5%.

As used herein, the term “antibody” refers to an immunoglobulin molecule which has the ability to specifically bind to a specific antigen. An antibody often comprises a variable region and a constant region in each of a heavy chain and a light chain. The variable regions of the heavy and light chains of antibodies contain a binding domain that interacts with an antigen. The constant regions of antibodies may mediate the binding of the immunoglobulin to host tissues or factors, including various cells of the immune system (such as effector cells) and components of the complement system such as C1q, the first component in the classical pathway of complement activation. Most antibodies have a heavy chain variable region (VH) and a light chain variable region (VL) that together form the portion of the antibody that binds to the antigen.

A “light chain variable region” (VL) or “heavy chain variable region” (VH) consists of four “framework” regions interrupted by three “complementarity determining regions” or “CDRs”. The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as LCDR1, LCDR2, and LCDR3; CDRs 1, 2, and 3 of a VH domain are also referred to herein, respectively, as HCDR1, HCDR2, and HCDR3.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR (also named Chothia and Kabat Combined CDR), in which each CDR is a composite of Chothia and Kabat CDR; the AbM definition used by Oxford Molecular's antibody modelling software; and, the contact definition of Martin et al. (world wide web bioinfo.org.uk/abs). Kabat provides a widely used numbering convention (Kabat numbering system) in which corresponding residues between different heavy chains or between different light chains are assigned the same number.

The present disclosure involves CDRs defined according to any of these numbering systems, although preferred embodiments involve Chothia and Kabat Combined defined CDRs.

TABLE 3
The definition methods for CDRs of an antibody
(see http://bioinf.org.uk/abs/)

| | Kabat | Chothia | Combined |
|---|---|---|---|
| LCDR1 | L24--L34 | L24--L34 | L24-L34 |
| LCDR2 | L50--L56 | L50--L56 | L50-L56 |
| LCDR3 | L89--L97 | L89--L97 | L89-L97 |
| HCDR1 | H31--H35 | H26--H32 | H26-H35 |
| HCDR2 | H50--H65 | H52--H56 | H50-H65 |
| HCDR3 | H95--H102 | H95--H102 | H95-H102 |

In Table 3, Laa-Lbb may refers to the amino acid sequence from position aa (according to Chothia numbering system) to position bb (according to Chothia numbering system) starting from the N-terminus of the antibody light chain; and Haa-Hbb may refer to the amino acid sequence from position aa (according to Chothia numbering system) to position bb (according to Chothia numbering system) starting from the N-terminus of the antibody heavy chain. For example, L24-L34 can refer to the amino acid sequence from position 24 to position 34 (according to Chothia numbering system) starting from the N-terminus of the antibody light chain; and H26-H32 may refer to the amino acid sequence from position 26 to position 32 (according to Chothia numbering system) starting from the N-terminus of the antibody heavy chain.

The term “antibody” as used herein should be understood in its broadest meaning, and includes monoclonal antibodies (including full-length monoclonal antibodies), polyclonal antibodies, antibody fragments, and multi-specific antibodies containing at least two different antigen binding regions (e.g., bispecific antibodies). The antibody may contain additional modifications, such as non-naturally occurring amino acids, mutations in Fc regions, and mutations in glycosylation sites. Antibodies also include post-translation modified antibodies, fusion proteins containing the antigenic determinants of the antibody, and immunoglobulin molecules containing any other modifications to antigen recognition sites, as long as these antibodies exhibit desired biological activity.

As used herein, the term “antigen binding fragment” of an antibody refers to one or more fragments of an antibody that retain the ability to specifically bind to an antigen (e.g., CEACAM5 or CEACAM6). It has been shown that the antigen binding function of an antibody can be performed by fragments of a full-length antibody.

Examples of antigen binding fragment of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, FUNDAMENTALIMMUNOLOGY (Paul ed., 3.sup.rd ed. 1993)); (iv) a Fd fragment consisting of the VH and CH1 domains; (v) a Fd′ fragment having VH and CH1 domains and one or more cysteine residues at the C-terminus of the CH1 domain; (vi) a Fv fragment consisting of the VL and VH domains of a single arm of an antibody, (vii) a dAb fragment (Ward et al., (1989) Nature 341:544-546), which consists of a VH domain; (viii) an isolated complementarity determining region (CDR); and (ix) a nanobody, a heavy chain variable region containing a single variable domain and two constant domains. Furthermore, although the two domains of the Fv fragment, V Land VH are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding fragment” of an antibody. Furthermore, the term also includes a “linear antibody” comprising a pair of tandem Fd segments (VH-CH1-VH-CH1), which forms an antigen binding region together with a complementary light chain polypeptide, and a modified version of any of the foregoing fragments, which retains antigen binding activity.

These antigen binding fragments can be obtained using conventional techniques known to those with skill in the art, and the fragments are screened for utility in the same manner as are intact antibodies.

As used herein, the term “binding” or “specifically binding” refers to a non-random binding reaction between two molecules, such as between an antibody and its target antigen. The binding specificity of an antibody can be determined based on affinity and/or avidity. The affinity, represented by the equilibrium constant for the dissociation of an antigen with an antibody (KD), is a measure for the binding strength between an antigenic determinant (epitope) and an antigen-binding site on the antibody: the lesser the value of the KD, the stronger the binding strength between an antigenic determinant (epitope) and the antibody. Alternatively, the affinity can also be expressed as the affinity constant (KA), which is 1/KD.

Avidity is the measure of the strength of binding between an antibody and the pertinent antigen. Avidity is related to both the affinity between an antigenic determinant (epitope) and its antigen binding site on the antibody and the number of pertinent binding sites present on the antibody. Typically, an antibody will bind with a dissociation constant (KD) of 10⁻⁵ to 10⁻¹² M or less, and preferably 10⁻⁷ to 10⁻¹² M or less and more preferably 10⁻⁸ to 10⁻¹² M, and/or with a binding affinity of at least 10⁷ M⁻¹, preferably at least 10⁸ M⁻¹, more preferably at least 10⁹ M⁻¹, such as at least 10¹² M⁻¹. Any K_{D} value greater than 10⁻⁴ M is generally considered to indicate non-specific binding. Specifically binding of an antibody to an antigen or antigenic determinant can be determined in any suitable manner known per se, including, for example, Scatchard analysis and/or competitive binding assays, such as radioimmunoassays (RIA), enzyme immunoassays (EIA), bio-layer interferometry (BLI) assay and sandwich competition assays, and the different variants thereof known per se in the art.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. The epitope defines the smallest binding site of an antibody and therefore is the specific target of the antibody or antigen binding fragment thereof.

As used herein, the term “sequence identity” refers to the extent to which two sequences (amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

As used herein, the term “monoclonal antibody” refers to an antibody obtained from a substantially homogeneous antibody population. That is, each antibody constituting the population are the same, except for possible naturally occurring mutations in small amount. Monoclonal antibodies are highly specific and are directed against a single antigen. The term “monoclonal antibody” herein is not limited to antibodies produced by hybridoma technology, and should not be interpreted as requiring production of antibodies by any specific method.

The term “bispecific antibody” is in the context of the present invention to be understood as an antibody having two different antigen-binding regions defined by different antibody sequences. This can be understood as different target binding but includes as well binding to different epitopes in one target.

As used herein, the term “tumor associated antigen” refers to an antigen that is differentially expressed in cancer cells compared to normal cells, and therefore can be used to target cancer cells.

As used herein, the term “vector” is intended to refer to a nucleic acid molecule capable of transporting another nucleic acid to which it has been linked.

As used herein, the term “host cell” refers to a cell into which an expression vector has been introduced.

The term “pharmaceutically acceptable” means that the carrier or adjuvant is compatible with the other ingredients of the composition and not substantially deleterious to the recipient thereof and/or that such carrier or adjuvant is approved or approvable for inclusion in a pharmaceutical composition for parenteral administration to humans.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptom of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the antibodies or compositions or conjugates disclosed herein to prevent or delay, to alleviate, or to arrest or inhibit development of the symptoms or conditions associated with diseases (e.g., cancers). The term “therapeutic effect” refers to the reduction, elimination, or prevention of the disease, symptoms of the disease, or side effects of the disease in the subject.

The term “effective amount” as used herein means the amount that, when administered to a subject for treating a disease, is sufficient to effect treatment for that disease.

The term “subject”, as used herein, refers to any mammalian subject for whom diagnosis, treatment, or therapy is desired. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc.

The terms “cyno CEACAM5”, “cynomolgus CEACAM5”, and “Cynomolgus macaques CEACAM5” are used interchangeably herein, and are refer to cynomolgus monkey CEACAM5. The term includes any CEACAM5 variants, isoforms and species homologs which are naturally expressed by cynomolgus monkey cells, or are expressed on cells transfected with genes or cDNA encoding the cynomolgus monkey CEACAM5 which are naturally expressed on cynomolgus monkey cells.

The terms “cyno CEACAM6”, “cynomolgus CEACAM6”, and “Cynomolgus macaques CEACAM6” are used interchangeably herein, and are refer to cynomolgus monkey CEACAM6. The term includes any CEACAM6 variants, isoforms and species homologs which are naturally expressed by cynomolgus monkey cells, or are expressed on cells transfected with genes or cDNA encoding the cynomolgus monkey CEACAM6 which are naturally expressed on cynomolgus monkey cells.

### Anti-CEACAM5/6 Dual-Binding Antibodies

In a first aspect, the invention provides an antibody that binds to both CEACAM5 and CEACAM6, or an antigen binding fragment thereof, wherein the antibody comprises a heavy chain variable region (VH) and a light chain variable region (VL), and wherein (1) the VH comprises the HCDRs 1-3 of a VH having the amino acid sequence set forth in SEQ ID NO: 26, and the VL comprises the LCDRs 1-3 of a VL having the amino acid sequence set forth in SEQ ID NO: 27; (2) the VH comprises the HCDRs 1-3 of a VH having the amino acid sequence set forth in SEQ ID NO: 7, and the VL comprises the LCDRs 1-3 of a VL having the amino acid sequence set forth in SEQ ID NO: 8; (3) the VH comprises the HCDRs 1-3 of a VH having the amino acid sequence set forth in SEQ ID NO: 17, and the VL comprises the LCDRs 1-3 of a VL having the amino acid sequence set forth in SEQ ID NO: 18; or (4) the VH comprises the HCDRs 1-3 of a VH having the amino acid sequence set forth in SEQ ID NO: 31, and the VL comprises the LCDRs 1-3 of a VL having the amino acid sequence set forth in SEQ ID NO: 32.

In some embodiments, the LCDRs 1-3 and HCDRs 1-3 are defined by EU Kabat definition/numbering system. In some embodiments, the LCDRs 1-3 and HCDRs 1-3 are defined by Chothia definition/numbering system. In some embodiments, the LCDRs 1-3 and HCDRs 1-3 are defined by AbM definition/numbering system. In some preferred embodiments, the LCDRs 1-3 and HCDRs 1-3 are defined by Kabat and Chothia combined numbering system.

In some embodiments, the antibody comprises a heavy chain variable region (VH) and a light chain variable region (VL), wherein (1) the VH comprises HCDRs 1-3 comprising the amino acid sequences set forth in SEQ ID NOs: 21, 22 and 23 respectively, and the VL comprises LCDRs 1-3 comprising the amino acid sequences set forth in SEQ ID NOs: 24, 5 and 25 respectively; (2) the VH comprises HCDRs 1-3 comprising the amino acid sequences set forth in SEQ ID NOs: 1, 2 and 3 respectively, and the VL comprises LCDRs 1-3 comprising the amino acid sequences set forth in SEQ ID NOs: 4, 5 and 6 respectively; (3) the VH comprises HCDRs 1-3 comprising the amino acid sequences set forth in SEQ ID NOs: 11, 12 and 13 respectively, and the VL comprises LCDRs 1-3 comprising the amino acid sequences set forth in SEQ ID NOs: 14, 15 and 16 respectively; or (4) the VH comprises HCDRs 1-3 comprising the amino acid sequences set forth in SEQ ID NOs: 1, 30 and 3 respectively, and the VL comprises LCDRs 1-3 comprising the amino acid sequences set forth in SEQ ID NOs: 4, 5 and 6 respectively.

In some embodiments, the CDRs are determined by Kabat and Chothia Combined system.

In some embodiments, the antibody comprises a heavy chain variable region (VH) and a light chain variable region (VL), wherein (1) the VH comprises an amino acid sequence having at least 80%, at least 85%, at least 90%, at least 95%, at least 98%, at least 99%, or 100% sequence identity to SEQ ID NO: 26, and the VL comprises an amino acid sequence having at least 80%, at least 85%, at least 90%, at least 95%, at least 98%, at least 99%, or 100% sequence identity to SEQ ID NO: 27; (2) the VH comprises an amino acid sequence having at least 80%, at least 85%, at least 90%, at least 95%, at least 98%, at least 99%, or 100% sequence identity to SEQ ID NO: 7, and the VL comprises an amino acid sequence having at least 80%, at least 85%, at least 90%, at least 95%, at least 98%, at least 99%, or 100% sequence identity to SEQ ID NO: 8; (3) the VH comprises an amino acid sequence having at least 80%, at least 85%, at least 90%, at least 95%, at least 98%, at least 99%, or 100% sequence identity to SEQ ID NO: 17, and the VL comprises an amino acid sequence having at least 80%, at least 85%, at least 90%, at least 95%, at least 98%, at least 99%, or 100% sequence identity to SEQ ID NO: 18; or (4) the VH comprises an amino acid sequence having at least 80%, at least 85%, at least 90%, at least 95%, at least 98%, at least 99%, or 100% sequence identity to SEQ ID NO: 31, and the VL comprises an amino acid sequence having at least 80%, at least 85%, at least 90%, at least 95%, at least 98%, at least 99%, or 100% sequence identity to SEQ ID NO: 32.

In some embodiments, the VH comprises a functional variant of the amino acid sequence as set forth in any one of SEQ ID NO: 26, 7, 17, and 31 formed by insertion, deletion and/or substitution of one or more amino acid(s) therein, provided that the antibody comprising the VH comprising the functional variant retains the ability of binding to both CEACAM5 and CEACAM6. In some embodiments, the VL comprises a functional variant of the amino acid sequence as set forth in any one of SEQ ID NOs: 27, 8, 18, and 32 formed by insertion, deletion and/or substitution of one or more amino acid(s) therein, provided that the antibody comprising the VL comprising the functional variant retains the ability of binding to both CEACAM5 and CEACAM6.

The functional variant comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the amino acid sequence of the parent polypeptide.

In the context of the functional variant, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-20, preferably 1-10, more preferably 1-7, still more preferably 1-5, and most preferably 1-2. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3, and/or FR4.

In some embodiments, the substitution of one or more amino acid(s) can be conservative substitution of one or more amino acid(s). Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In preferred embodiments, the invention provides an antibody that binds to both CEACAM5 and CEACAM6, or an antigen binding fragment thereof, wherein the antibody comprises a heavy chain variable region (VH) and a light chain variable region (VL), and wherein
(1) the VH comprises an amino acid sequence set forth in SEQ ID NO: 26, and the VL comprises an amino acid sequence set forth in SEQ ID NO: 27;
(2) the VH comprises an amino acid sequence set forth in SEQ ID NO: 7, and the VL comprises an amino acid sequence set forth in SEQ ID NO: 8;
(3) the VH comprises an amino acid sequence set forth in SEQ ID NO: 17, and the VL comprises an amino acid sequence set forth in SEQ ID NO: 18; or
(4) the VH comprises an amino acid sequence set forth in SEQ ID NO: 31, and the VL comprises an amino acid sequence set forth in SEQ ID NO: 32.

In some embodiments, the antibody comprises a heavy chain (HC) and a light chain (LC), wherein (1) the HC comprises an amino acid sequence having at least 80%, at least 85%, at least 90%, at least 95%, at least 98%, at least 99%, or 100% sequence identity to SEQ ID NO: 28, and the LC comprises an amino acid sequence having at least 80%, at least 85%, at least 90%, at least 95%, at least 98%, at least 99%, or 100% sequence identity to SEQ ID NO: 29; (2) the HC comprises an amino acid sequence having at least 80%, at least 85%, at least 90%, at least 95%, at least 98%, at least 99%, or 100% sequence identity to SEQ ID NO: 9, and the LC comprises an amino acid sequence having at least 80%, at least 85%, at least 90%, at least 95%, at least 98%, at least 99%, or 100% sequence identity to SEQ ID NO: 10; (3) the HC comprises an amino acid sequence having at least 80%, at least 85%, at least 90%, at least 95%, at least 98%, at least 99%, or 100% sequence identity to SEQ ID NO: 19, and the LC comprises an amino acid sequence having at least 80%, at least 85%, at least 90%, at least 95%, at least 98%, at least 99%, or 100% sequence identity to SEQ ID NO: 20; or (4) the HC comprises an amino acid sequence having at least 80%, at least 85%, at least 90%, at least 95%, at least 98%, at least 99%, or 100% sequence identity to SEQ ID NO: 33, and the LC comprises an amino acid sequence having at least 80%, at least 85%, at least 90%, at least 95%, at least 98%, at least 99%, or 100% sequence identity to SEQ ID NO: 34.

In some embodiments, the heavy chain comprises a functional variant of the amino acid sequence as set forth in any one of SEQ ID NOs: 28, 9, 19, and 33 formed by insertion, deletion and/or substitution of one or more amino acid(s) therein, provided that the antibody comprising the heavy chain comprising the functional variant retains the ability of binding to both CEACAM5 and CEACAM6. In some embodiments, the light chain comprises a functional variant of the amino acid sequence as set forth in any one of any one of SEQ ID NOs: 29, 10, 20, and 34 formed by insertion, deletion and/or substitution of one or more amino acid(s) therein, provided that the antibody comprising the light chain comprising the functional variant retains the ability of binding to both CEACAM5 and CEACAM6.

The functional variant comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the amino acid sequence of the parent polypeptide.

In some embodiments, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-50, preferably 1-20, more preferably 1-10, still more preferably 1-5. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3 and/or FR4; and/or constant regions, e.g., CL, CH1, CH2 and/or CH3.

In some embodiments, the substitution of one or more amino acid(s) can be conservative substitution of one or more amino acid(s). Examples of conservative substitutions are as described above.

In preferred embodiments, the antibody comprises a heavy chain (HC) and a light chain (LC), wherein:
(1) the HC comprises an amino acid sequence set forth in SEQ ID NO: 28, and the LC comprises an amino acid sequence set forth in SEQ ID NO: 29;
(2) the HC comprises an amino acid sequence set forth in SEQ ID NO: 9, and the LC comprises an amino acid sequence set forth in SEQ ID NO: 10;
(3) the HC comprises an amino acid sequence set forth in SEQ ID NO: 19, and the LC comprises an amino acid sequence set forth in SEQ ID NO: 20; or
(4) the HC comprises an amino acid sequence set forth in SEQ ID NO: 33, and the LC comprises an amino acid sequence set forth in SEQ ID NO: 34.

In some embodiments, the antibody is a murine antibody, a chimeric antibody, a humanized antibody, or a human antibody.

Based on the amino acid sequence of heavy chain constant regions of the antibody, a immunoglobulin molecule can be divided into five classes (isotypes): IgA, IgD, IgE, IgG, and IgM, and can be further divided into different subtypes, such as IgG1, IgG2, IgG3, IgG4, IgA1, IgA2, etc. The light chain of the antibody can be classified as a lambda (λ) chain or a kappa (κ) chain, based on the amino acid sequence of the light chain. The antibodies disclosed herein can be of any classes or subtypes above.

In some embodiments, the antibody can be of an isotype selected from the group consisting of IgG, IgA, IgM, IgE and IgD. In some embodiments, the antibody can be of a subtype selected from the group consisting of IgG1, IgG2, IgG3, and IgG4. In a preferred embodiment, the antibody is an IgG1 antibody.

The antibody disclosed herein can be an intact antibody or the antigen binding fragment thereof. The antigen binding fragment can be any fragments of the antibody that retain the ability to bind to both CEACAM5 and CEACAM6. Examples of antigen binding fragments include but are not limited to a Fab fragment; a F(ab′)₂ fragment; a Fab′ fragment; a Fd fragment; a Fd′ fragment; a Fv fragment; a scFv fragment; a dAb fragment; an isolated complementarity determining region (CDR); a nanobody; a linear antibody comprising a pair of tandem Fd segments (VH-CH1-VH-CH1), and a modified version of any of the foregoing fragments, which retains antigen binding activity.

In some embodiments, the antigen binding fragment can be selected from the group consisting of Fab, Fab′, F(ab′)₂, Fv, scFv, and ds-scFv. In a preferred embodiment, the antigen binding fragment is Fab or scFv.

In some embodiments, the antibody comprises an Fc region. In some embodiments, the Fc region may be of any isotype, including, but not limited to, IgG1, IgG2, IgG3 and IgG4, and may comprise one or more mutations or modifications. In one embodiment, the Fc region is of IgG1 isotype or derived therefrom, optionally with one or more mutations or modifications. In one embodiment, the Fc region is human IgG1 Fc.

In one embodiment, the Fc region is effector-function-deficient. For example, the Fc region may be of an IgG1 isotype, or a non-IgG1 type, e.g., IgG2, IgG3 or IgG4, which has been mutated such that the ability to mediate effector functions, such as ADCC, has been reduced or even eliminated. Such mutations have e.g., been described in Dall'Acqua W F et al., J Immunol. 177 (2): 1129-1138 (2006) and Hezareh M, J Virol.; 75 (24): 12161-12168 (2001). In some embodiments, the Fc region of the antibody comprises a wild type IgG1 Fc with L234A, L235A and G237A mutations.

In some embodiments, the antibody is mutated at one or more post-translational modifications sites. In one embodiment, the Fc region comprises a mutation removing the acceptor site for Asn-linked glycosylation or is manipulated to eliminate the effector function of the antibody.

Post Translational Modification (PTM) is widely observed in proteins expressed in mammalian cells. Except for conserved PTM sites in antibody, e.g. conserved N-glycosylation site on IgG1 antibody CH2 domain, other PTM sites occurred within antigen binding sites of antibody (i.e., CDR regions) may reduce antigen binding activity or reduce chemical stability. For example, deamidation or isomerization may make the molecules unstable and heterogenous. To reduce the sequence liability, the PTM motifs could be removed by mutations. The VH or VL sequences were scanned by the presence of PTM motifs, e.g., isomerization motifs (e.g., DG). Then the “hotspot” residue (e.g., D or G in DG motif) was mutated to either the counterpart residue in germline sequence or other residue with similar biophysical properties.

In some embodiments, the antibody is a monoclonal antibody, a bi-specific or a multi-specific antibody. In some embodiments, the antibody is monovalent, bivalent or multivalent.

### Bispecific Antibodies

In a second aspect, the present application provides a bispecific or a multi-specific antibody. In some embodiments, the antibody is a bispecific antibody which further comprises a second antigen binding region binding to a second antigen. In some embodiments, the second antigen can be a tumor associated antigen, an immune checkpoint molecule or an immune cell antigen.

Many tumor associated antigens associated with specific cancers have been identified in the art. In some embodiments, tumor-associated antigens are antigens that can potentially stimulate an obvious tumor-specific immune response. Some of these antigens are encoded by normal cells, but not necessarily expressed by normal cells. These antigens can be characterized as those that are usually silent (i.e., not expressed) in normal cells, those that are expressed only during certain stages of differentiation, and those that are expressed over time, such as embryonic and fetal antigens. Other cancer antigens are encoded by mutant cell genes such as oncogenes (e.g. activated ras oncogene), suppressor genes (e.g. mutant p53), and fusion proteins produced by internal deletions or chromosomal translocations. Other cancer antigens can be encoded by viral genes, such as those carried on RNA and DNA tumor viruses. Many other tumor associated antigens and antibodies against them are known and/or commercially available, and can also be produced by those skilled in the art.

Examples of tumor associated antigens include but are not limited to 5T4, alphafetoprotein, CA-125, mesothelin, CD19, CD20, CD22, CD23, CD30, CD33, CD40, CD56, CD79, CD78, CD123, CD138, c-Met, CSPG4, IgM, C-type lectin-like molecule 1 (CLL-1), EGFR, EGFRvIII, epithelial tumor antigen, ERBB2, FLT3, folate binding protein, GD2, GD3, HIV-1 envelope glycoprotein gp41, HIV-1 envelope glycoprotein gp120, melanoma-associated antigen, CD200R1, MUC-1, mutated p53, mutated ras, ROR1, VEGFR2, and combinations thereof.

In some embodiments, the second antigen is a T-cell antigen. In some embodiments, the T-cell antigen can be selected from the group consisting of T cell receptor (TCR), CD3, CD4, CD8, CD16, CD25, CD28, CD44, CD62L, CD69, ICOS, 41-BB (CD137), and NKG2D or any combination thereof. 8, CD44, CD62L, CD69, ICOS, 41-BB (CD137), and NKG2D or any combination thereof.

In some embodiments, the second antigen is an immune checkpoint molecule. In some embodiments, the immune checkpoint molecule can be selected from the group consisting of PD-1, PD-L1, CTLA-4, and the like.

In some embodiments, the bispecific antibody comprises a single polypeptide chain or multiple (e.g. two or four) polypeptide chain comprising the first antigen binding region and the second antigen binding region, and optionally an Fc region.

The Fc region may be of any isotype, including, but not limited to, IgG1, IgG2, IgG3 and IgG4, and may comprise one or more mutations or modifications. In one embodiment, the Fc region is of IgG1 isotype or derived therefrom, optionally with one or more mutations or modifications. In one embodiment, the Fc region is human IgG1 Fc.

In one embodiment, the Fc region is effector-function-deficient. For example, the Fc region may be of an IgG1 isotype, or a non-IgG1 type, e.g., IgG2, IgG3 or IgG4, which has been mutated such that the ability to mediate effector functions, such as ADCC, has been reduced or even eliminated. Such mutations have e.g., been described in Dall'Acqua W F et al., J Immunol. 177 (2): 1129-1138 (2006) and Hezareh M, J Virol.; 75 (24): 12161-12168 (2001).

In one embodiment, the Fc region comprises a mutation removing the acceptor site for Asn-linked glycosylation or is otherwise manipulated to change the glycosylation properties. For example, in an IgG1 Fc region, an N297Q mutation can be used to remove an Asn-linked glycosylation site. Accordingly, in a specific embodiment, Fc region comprise an IgG1 wildtype sequence with an N297Q mutation. For example, in an IgG1 Fc region, an N297Q mutation can be used to remove an Asn-linked glycosylation site. Accordingly, in a specific embodiment, Fc region comprise an IgG1 wildtype sequence with an N297Q mutation.

In a further embodiment, the Fc region is glyco-engineered to reduce fucose and thus enhance ADCC, e.g., by addition of compounds to the culture media during antibody production as described in US2009317869 or as described in van Berkel et al. (2010) Biotechnol. Bioeng. 105:350 or by using FUT8 knockout cells, e.g., as described in Yamane-Ohnuki et al. (2004) Biotechnol. Bioeng 87:614. ADCC may alternatively be optimized using the method described by Umaña et al. (1999) Nature Biotech 17:176. In a further embodiment, the Fc region has been engineered to enhance complement activation, e.g., as described in Natsume et al. (2009) Cancer Sci. 100:2411.

### Nucleic Acids

In a third aspect, the invention provides a nucleic acid comprising a nucleotide sequence encoding the anti-CEACAM5/6 dual-binding antibody or the antigen binding fragment thereof disclosed herein or the bispecific antibody or the antigen binding fragment thereof disclosed herein.

The term “polynucleotide” or “nucleic acid” includes both single-stranded and double-stranded nucleotide polymers. The nucleotides comprising the nucleic acid can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

For example, the invention provides nucleic acid molecules encoding any one of the heavy chain variable region sequences disclosed herein. The invention also provides nucleic acid molecules that are at least 90%, at least 95%, at least 98% or at least 99% identical to nucleic acids encoding any one of the heavy chain variable region sequences disclosed herein.

For example, the invention provides nucleic acid molecules encoding any one of the light chain variable region sequences disclosed herein. The invention also provides nucleic acid molecules that are at least 90%, at least 95%, at least 98% or at least 99% identical to nucleic acids encoding any one of the light chain variable region sequences disclosed herein.

For example, the invention provides nucleic acid molecules encoding: (i) any one of the heavy chain variable region sequences disclosed herein and (ii) any one of the light chain variable region sequences disclosed herein. The invention also provides nucleic acid molecules that are at least 90%, at least 95%, at least 98% or at least 99% identical to nucleic acids encoding: (i) any one of the heavy chain variable region sequences disclosed herein and (ii) any one of the light chain variable region sequences disclosed herein.

For example, the invention provides nucleic acid molecules encoding a heavy chain variable region sequence that comprises the CDR sequences of any one of the heavy chain variable region sequences disclosed herein.

In some embodiments, the invention provides nucleic acid molecules encoding a heavy chain variable region sequence that comprises any one of the groups of three CDR sequences disclosed herein.

The invention also provides nucleic acid molecules that encode a heavy chain variable region sequence that comprises CDR sequences that are at least 90%, at least 95%, at least 98% or at least 99% identical to the CDR sequences of any one of the heavy chain variable region sequences disclosed herein.

In some embodiments, the invention provides nucleic acid molecules that encode a heavy chain variable region sequence that comprises CDR1, CDR2 and CDR3 sequences that are at least 90%, at least 95%, at least 98% or at least 99% identical to the CDR1, CDR2 and CDR3, respectively, of any one of the groups of three CDR sequences disclosed herein.

For example, the invention provides nucleic acid molecules encoding a light chain variable region sequence that comprises the CDR sequences of any one of the light chain variable region sequences disclosed herein.

In some embodiments, the invention provides nucleic acid molecules encoding a light chain variable region sequence that comprises any one of the groups of three CDR sequences disclosed herein.

The invention also provides nucleic acid molecules that encode a light chain variable region sequence that comprises CDR sequences that are at least 90%, at least 95%, at least 98% or at least 99% identical to the CDR sequences of any one of the light chain variable region sequences disclosed herein.

In some embodiments, the invention provides nucleic acid molecules that encode a light chain variable region sequence that comprises CDR1, CDR2 and CDR3 sequences that are at least 90%, at least 95%, at least 98% or at least 99% identical to the CDR1, CDR2 and CDR3, respectively, of any one of the groups of three CDR sequences disclosed herein.

For example, the invention provides nucleic acid molecules encoding: (i) a heavy chain variable region sequence that comprises the CDR sequences of any one of the heavy chain variable region sequences disclosed herein and (ii) a light chain variable region sequence that comprises the CDR sequences of any one of the light chain variable region sequences disclosed herein. In some embodiments, the invention provides nucleic acid molecules encoding (i) a heavy chain variable region sequence that comprises any one of the groups of three CDR sequences disclosed herein and (ii) a light chain variable region sequence that comprises any one of the groups of three CDR sequences disclosed herein. The invention also provides nucleic acid molecules that encode: (i) a heavy chain variable region sequence that comprises CDR sequences that are at least 90%, at least 95%, at least 98% or at least 99% identical to the CDR sequences of any one of the heavy chain variable region sequences disclosed herein and (ii) a light chain variable region sequence that comprises CDR sequences that are at least 90%, at least 95%, at least 98% or at least 99% identical to the CDR sequences of any one of the light chain variable region sequences disclosed herein. In some embodiments, the invention provides nucleic acid molecules that encode (i) a heavy chain variable region sequence that comprises CDR1, CDR2 and CDR3 sequences that are at least 90%, at least 95%, at least 98% or at least 99% identical to the CDR1, CDR2 and CDR3, respectively, of any one of the groups of three CDR sequences disclosed herein and (ii) a light chain variable region sequence that comprises CDR1, CDR2 and CDR3 sequences that are at least 90%, at least 95%, at least 98% or at least 99% identical to the CDR1, CDR2 and CDR3, respectively, of any one of the groups of three CDR sequences disclosed herein.

In some embodiments, the nucleic acid is ribonucleic acid (RNA) or deoxyribonucleic acid (DNA). In some embodiments, the invention provides a ribonucleic acid (RNA) comprising a nucleotide sequence encoding the anti-CEACAM5/6 dual-binding antibody or the antigen binding fragment thereof disclosed herein or the bispecific antibody or the antigen binding fragment thereof disclosed herein. In some embodiments, the invention provides a deoxyribonucleic acid (DNA) comprising a deoxynucleotide sequence encoding the anti-CEACAM5/6 dual-binding antibody or the antigen binding fragment thereof disclosed herein or the bispecific antibody or the antigen binding fragment thereof disclosed herein.

In some embodiments, the deoxyribonucleic acid (DNA) may be introduced into the cells of a human body in vivo. In some embodiments, the deoxyribonucleic acid (DNA) of the invention is comprised in a vector or a delivering agent. In some embodiments, the deoxyribonucleic acid (DNA) of the invention is integrated into the genome of a cell.

In some embodiments, the ribonucleic acid (RNA) may be introduced into the cells of a human body in vivo. In some embodiments, the ribonucleic acid (RNA) of the invention is comprised in a vector or a delivering agent.

In some specific embodiments, the ribonucleic acid (RNA) comprising a nucleotide sequence encoding the anti-CEACAM5/6 dual-binding antibody or the antigen binding fragment thereof disclosed herein or the bispecific antibody or the antigen binding fragment thereof disclosed herein is a mRNA. In some embodiments, the mRNA of the invention is comprised in a vector or a delivering system (such as lipidosome). In some embodiments, the mRNA may be introduced into the cells of a human body in vivo via a vector or a delivering system (such as lipidosome) and expresses a CEACAM5 antibody of the invention in vivo.

### Vectors

In the fourth aspect, the invention further provides a vector, which comprises the nucleic acid comprising a nucleotide sequence encoding the anti-CEACAM5/6 dual-binding antibody or the antigen binding fragment thereof disclosed herein or the bispecific antibody or the antigen binding fragment thereof disclosed herein.

In some embodiments, the vector is a recombinant expression vector capable of expressing a polypeptide comprising a heavy or light chain variable region of an anti-CEACAM5/6 dual-binding antibody. For example, the invention provides recombinant expression vectors comprising any of the nucleic acid molecules mentioned above.

Any vector may be suitable for the present disclosure. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof. Suitable exemplary vectors include e.g., pGAR, pBABE-puro, pBABE-neo largeTcDNA, pBABE-hygro-hTERT, pMKO.1 GFP, MSCV-IRES-GFP, pMSCV PIG (Puro IRES GFP empty plasmid), pMSCV-loxp-dsRed-loxp-eGFP-Puro-WPRE, MSCV IRES Luciferase, pMIG, MDH1-PGK-GFP_2.0, TtRMPVIR, pMSCV-IRES-mCherry FP, pRetroX GFP T2A Cre, pRXTN, pLncEXP, and pLXIN-Luc.

A recombinant expression vector may be any suitable recombinant expression vector. Suitable vectors comprise those designed for propagation and expansion or for expression or both, such as plasmids and viruses. For example, a vector may be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also may be used. Examples of plant expression vectors useful in the context of the disclosure comprise pBI01, pBI101.2, pBI101.3, pBI121 and pBIN19 (Clontech). Examples of animal expression vectors useful in the context of the disclosure comprise pcDNA, pEUK-Cl, pMAM, and pMAMneo (Clontech).

Recombinant expression vectors may be prepared using standard recombinant DNA techniques described in, for example, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Constructs of expression vectors, which are circular or linear, may be prepared to contain a replication system functional in a prokaryotic or eukaryotic host cell. Replication systems may be derived, e.g., from ColE1, 2μ plasmid, λ, SV40, bovine papilloma virus, and the like.

The vector of the invention may be introduced into a cell. In some embodiments, the vector of the invention may be introduced into a cell in vitro or ex vivo. Optionally, the cell introduced with the vector may subsequently be administered into the body of a subject. In some embodiments, the vector of the invention may be introduced into a cell in vivo.

For example, the vector may be an adenoviral vector comprising a nucleotide sequence encoding the anti-CEACAM5/6 dual-binding antibody or the antigen binding fragment thereof disclosed herein or the bispecific antibody or the antigen binding fragment thereof disclosed herein. The vector may be administered into the body of a subject, and then enter into a cell of the subject in vivo, thereby the nucleotide sequence encoding the anti-CEACAM5/6 dual-binding antibody or the antigen binding fragment thereof disclosed herein or the bispecific antibody or the antigen binding fragment thereof disclosed herein is integrated into the genome of the cell, and subsequently the cell expresses the anti-CEACAM5/6 dual-binding antibody or the antigen binding fragment thereof disclosed herein.

### Host Cells

In the fifth aspect, the invention further provides a host cell comprising the nucleic acid disclosed herein or the vector disclosed herein.

Any cell may be used as a host cell for the nucleic acids or the vectors of the present disclosure. In some embodiments, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. aeruginosa; and Streptomyces. In some embodiments, the cell is a human cell. In some embodiments, the cell is an immune cell. In some embodiments, host cells include, for example, CHO cells, such as CHOS cells and CHOK1 cells, or HEK293 cells, such as HEK293A, HEK293T and HEK293FS.

The host cell of the invention is prepared by introducing the vector disclosed herein or the nucleic acid disclosed herein in vitro or ex vivo. The host cell of the invention may be administered into the body of a subject, and the host cell expresses the anti-CEACAM5/6 dual-binding antibody or the antigen binding fragment thereof disclosed herein in vivo so as to treat the diseases disclosed herein.

The invention further provides host cells into which any of the vectors mentioned above have been introduced. The invention further provides methods of producing the antibodies and antibody fragments of the invention by culturing the host cells under conditions permitting production of the antibodies or antibody fragments, and recovering the antibodies and antibody fragments so produced.

### Conjugates and Antibody-Drug Conjugates

The antibody or antigen binding fragment of the invention can be attached to a chemical moiety to form a conjugate.

In the context of the present disclosure, a “conjugate” is an antibody or antibody fragment (such as an antigen-binding fragment) covalently linked to a chemical moiety. The chemical moiety can be, for example, a drug, toxin, therapeutic agent, detectable label, protein, nucleic acid, lipid, nanoparticle, carbohydrate or recombinant virus. An antibody conjugate is often referred to as an “immunoconjugate”. When the conjugate comprises an antibody linked to a drug (e.g., a cytotoxic agent), the conjugate is often referred to as an “antibody-drug conjugate” or “ADC.”

The term “conjugated” or “linked” may refer to making two polypeptides into one contiguous polypeptide molecule. In one embodiment, an antibody is joined to a chemical moiety. In another embodiment, an antibody joined to a chemical moiety is further joined to a lipid or other molecule to a protein or peptide to increase its half-life in the body. The linkage can be either by chemical or recombinant means. In one embodiment, the linkage is chemical, wherein a reaction between the antibody moiety and the chemical moiety has produced a covalent bond formed between the two molecules to form one molecule. A peptide linker (short peptide sequence) can optionally be included between the antibody and the chemical moiety.

A chemical moiety can be linked to the antibody of the invention using any number of means known to those of skill in the art. Both covalent and noncovalent attachment means may be used. The procedure for attaching a chemical moiety to the antibody varies according to the chemical structure of the chemical moiety. Polypeptides typically contain a variety of functional groups; such as carboxylic acid (COOH), free amine (—NH2) or sulfhydryl (—SH) groups, which are available for reaction with a suitable functional group on an antibody to result in the binding of the chemical moiety. Alternatively, the antibody is derivatized to expose or attach additional reactive functional groups. The derivatization may involve attachment of any of a number of known linker molecules. The linker can be any molecule used to join the antibody to the chemical moiety. The linker is capable of forming covalent bonds to both the antibody and to the chemical moiety. Suitable linkers are well known to those of skill in the art and include, but are not limited to, straight or branched-chain carbon linkers, heterocyclic carbon linkers, or peptide linkers. Where the antibody and the chemical moiety are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the chemical moiety from the antibody when the immunoconjugate has reached its target site. Therefore, in these circumstances, immunoconjugates will comprise linkages that are cleavable in the vicinity of the target site.

Cleavage of the linker to release the chemical moiety from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, labels (such as enzymes or fluorescent molecules), drugs, toxins, and other agents to antibodies one skilled in the art will be able to determine a suitable method for attaching a given agent to an antibody or other polypeptide.

The antibodies disclosed herein can be derivatized or linked to another molecule (such as another peptide or protein). In general, the antibodies or portion thereof is derivatized such that the binding to the target antigen is not affected adversely by the derivatization or labeling. For example, the antibody can be functionally linked (by chemical coupling, genetic fusion, noncovalent association or otherwise) to one or more other molecular entities, such as another antibody (for example, a bi-specific antibody or a diabody), a detection agent, a pharmaceutical agent, and/or a protein or peptide that can mediate association of the antibody or antibody portion with another molecule (such as a strep tavidin core region or a polyhistidine tag).

One type of derivatized antibody is produced by cross-linking two or more antibodies (of the same type or of different types). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

In some embodiments of the conjugate disclosed herein, the chemical moiety is selected from the group consisting of a therapeutic agent, a detectable moiety, and an immune stimulatory molecule. In some embodiments, the therapeutic agent includes but is not limited to immunomodulators, radioactive compounds, enzymes (for example perforin), chemotherapeutic agents (for example cis-platin), or a toxin. In some embodiments, the therapeutic agent can be such as maytansine, geldanamycin, tubulin inhibitors such as tubulin binding agents (e.g., auristatins), or minor groove binding agents such as calicheamicin.

Other suitable therapeutic agents include such as, small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E vincristine and doxorubicin; peptide cytotoxins, i.e. proteins or fragments thereof with the ability to kill mammalian cells, for example, ricin, diphtheria toxin, pseudomonas bacterial exotoxin A, DNase and RNase; radio-nuclides, i.e. unstable isotopes of elements which decay with the concurrent emission of one or more of α or β particles, or γ rays, for example, iodine-131, rhenium-186, indium-111, yttrium-90, bismuth-210, bismuth-213, actinium-225 and astatine-213; chelating agents may be used to facilitate the association of these radionuclides to the molecules, or multimers thereof.

In some embodiments, the detectable moiety can be selected from the group consisting of biotin, streptavidin, an enzyme or catalytically active fragment thereof, a radionuclide, a nanoparticle, a paramagnetic metal ion, or a fluorescent, phosphorescent, or chemiluminescent molecule. A detectable moiety for diagnostic purposes includes for instance, fluorescent labels, radiolabels, enzymes, nucleic acid probes and contrast reagents.

The antibody can be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP) and yellow fluorescent protein (YFP).

The antibody or antigen binding fragment can also be conjugated with enzymes that are useful for detection, such as horseradish peroxidase, β-galactosidase, luciferase, alkaline phosphatase, glucose oxidase and the like. When a bispecific antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. The bispecific antibody or antigen binding fragment may also be conjugated with biotin, and detected through indirect measurement of avidin or streptavidin binding. It should be noted that the avidin itself can be conjugated with an enzyme or a fluorescent label.

The antibody may be fused to a self-labelling protein tag (e.g. HaloTag). For example, the protein tag could be cloned at the end of a constant region. HaloTag is a self-labelling protein tag derived from a bacterial enzyme (a haloalkane dehalogenase), designed to covalently bind to a synthetic ligand. In some instances, the synthetic ligand comprises a chloroalkane linker attached to a fluorophore, such as a near-infrared fluorophore (Los et al. (2008) ACS Chem Biol. 3 (6): 373-82). The antibody may be labeled with a magnetic agent, such as gadolinium. Antibodies can also be labeled with lanthanides (such as europium and dysprosium), and manganese.

Paramagnetic particles, such as superparamagnetic iron oxide, are also of use as labels. The bispecific antibody may also be labeled with a predetermined polypeptide epitope recognized by a secondary reporter (such as leucine zipper pair sequences, binding sites for secondary antibodies, metal binding domains, epitope tags). In some embodiments, labels are attached by spacer arms of various lengths to reduce potential steric hindrance.

The antibody can also be labeled with a radiolabeled amino acid. The radiolabel may be used for both diagnostic and therapeutic purposes. For instance, the radiolabel may be used to detect expression of a target antigen by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

In some embodiments, the immune stimulatory molecule is an immune effector molecule which stimulates immune response. For example, the immune stimulatory molecule can be cytokines such as IL-2 and IFN-γ, chemokines such as IL-8, platelet factor 4, melanoma growth stimulatory protein, complement activators; viral/bacterial protein domains, or viral/bacterial peptides.

In some embodiments, the antibody or antigen binding fragment of the invention is attached to a fluorescent label, radiolabel or a drug moiety. In some embodiments, the antibody or antigen binding fragment of the invention is attached to a drug moiety to form an antibody-drug conjugate (ADC).

Accordingly, in the sixth aspect, the invention provides an antibody-drug conjugate (ADC), wherein the ADC comprises the antibody or the antigen-binding fragment thereof of the first aspect of the invention or the bi-specific antibody of the second aspect of the invention, and a drug moiety conjugated thereto via a linker.

The terms “drug moiety”, “drug payload”, “therapeutic molecule”, “therapeutic payload”, “therapeutic agents”, “therapeutic moieties”, as used interchangeably herein, refers to a chemical or biological moiety that is conjugated to a dual-binding antibody that binds to CEACAM5 and CEACAM6, or an antigen binding fragment thereof.

Examples of drugs that may be used in ADCs, i.e., drugs that may be conjugated to the antibodies, are provided below, and include antibiotics, DNA synthesis inhibitors, RNA polymerase II inhibitors, and RNA spliceosome inhibitors, microtubule inhibitors, antitumor antibiotics, immunomodulating agents, gene therapy vectors, alkylating agents, antiangiogenic agents, antimetabolites, boron-containing agents, chemoprotective agents, hormone agents, glucocorticoids, photoactive therapeutic agents, oligonucleotides, radioactive isotopes, radiosensitizers, topoisomerase inhibitors (such as topoisomerase I inhibitors), tyrosine kinase inhibitors, and combinations thereof.

In some embodiments, the drug moiety is selected from the group consisting of microtubule inhibitors, antibiotics, DNA synthesis inhibitors, topoisomerase inhibitors, RNA polymerase II inhibitors, and RNA spliceosome inhibitors.

In some embodiments, the drug moiety is microtubule inhibitors. In some specific embodiments, the drug moiety is auristatins. In some specific embodiments, the drug moiety is maytansines. In some specific embodiments, the drug moiety is tubulysins. In some specific embodiments, the drug moiety is cryptophycins. In some specific embodiments, the drug moiety is rhizoxin.

In some embodiments, the drug moiety is antibiotics. In some specific embodiments, the drug moiety is calicheamicins. In some specific embodiments, the drug moiety is doxorubicin. In some specific embodiments, the drug moiety is anthracyclines.

In some embodiments, the drug moiety is DNA synthesis inhibitors. In some specific embodiments, the drug moiety is duocarmycins. In some specific embodiments, the drug moiety is PBDs (pyrrolobenzodiazepines). In some specific embodiments, the drug moiety is IGNs (indolinobenzodiazepines).

In some embodiments, the drug moiety is topoisomerase inhibitors. In some specific embodiments, the drug moiety is camptothecin analogs.

In some embodiments, the drug moiety is RNA polymerase II inhibitors. In some specific embodiments, the drug moiety is Amanitins.

In some embodiments, the drug moiety is RNA spliceosome inhibitors selected from a group consisting of spliceostatins and thailanstatins.

In some embodiments, maytansinoids (DM1, DM2, DM3, DM4, maytansine and ansamitocins) and their analogs is preferred.

In some embodiments, auristatins (MMAE, MMAF, MMAD and ansamitocins) and their analogs is preferred.

In some embodiments, PBDs (pyrrolobenzodiazepines, or pyrrolo[2,1-c][1,4]-benzodiazepines, such as SG3199) are preferred. PBDs are sequence-selective DNA alkylating antibiotics with significant antitumor properties. PBDs have the ability to recognize and bond to specific sequences in DNA; one such sequence is is PuGPu (Purine-Guanine-Purine). PBDs also can bond to PuGPy (Purine-Guanine-Pyrimidine) or PyGPu sequences, over PyGPy sequences.

In some embodiments, the PBDs drug moiety is SG3199, which is a cytotoxic DNA minor groove interstrand crosslinking pyrrolobenzodiazepine (PBD) dimer.

In some embodiments, camptothecin analogs is preferred. Camptothecin analogs is a kind of DNA topoisomerase I inhibitor. Unlike monomethyl auristatin E, camptothecin-based therapies do not cause peripheral neuropathy clinically, suggesting that SGN-CD30C may have the potential to avoid one of the most common adverse events associated with BV.

In some specific embodiments, the camptothecin drug moiety is 7-ethyl-10-hydroxycamptothecin (also known as SN38).

In some specific embodiments, the camptothecin drug moiety is Dxd, a potent topoisomerase I inhibitor, or its analogs or derivatives.

In some specific embodiments, the camptothecin drug moiety is exatecan (also known as DX-8951).

In some embodiments, calicheamicin is preferred, which is an antitumor antibiotic and a cytotoxic agent that causes double-strand DNA breaks. N-Acetyl calicheamicin is a derivative of calicheamicin and is a potent enediyne antitumor antibiotic. In other embodiments, the drug moiety is doxorubicin or its analogs or derivatives.

The one or more drug moieties (e.g., therapeutic agents and/or diagnostic agents) may be indirectly conjugated to the anti-CEACAM5/6 dual-binding antibodies (e.g. by way of a linker with direct covalent or non-covalent interactions). Linkers can be chemical linking agents, such as homobifunctional and heterobifunctional cross-linkers, which are available from many commercial sources.

In some embodiments, the linker comprises a cleavable linker or a non-cleavable linker.

Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the antibody remains active. Alternatively, linkers can be substantially resistant to cleavage (e.g., stable linker or non-cleavable linker).

In some embodiments, the linker is an acid-labile linker. In some embodiments, the linker is a photo-labile linker. In some embodiments, the linker is a protease-sensitive linker. In some embodiments, the linker is a hydrazone linker. In some embodiments, the linker is an esterase cleavable linker. In some embodiments, the linker is a dimethyl linker. In some embodiments, the linker is a disulfide-containing linker. In some embodiments, the linker is a hydrophilic linker. In some embodiments, the linker is a procharged linker. In some embodiments, the linker is an acid-based linker.

In some embodiments, the linker comprises an acid-labile linker. In some embodiments, the linker comprises a hydrophilic linker. In some embodiments, the linker comprises a protease-sensitive linker. In some embodiments, the linker comprises photolabile linker. In some embodiments, the linker comprises a hydrazone linker. In some embodiments, the linker comprises a dimethyl linker. In some embodiments, the linker comprises a disulfide-containing linker.

In some embodiments, the linker may comprise an amino acid unit. In one such embodiment, the amino acid unit allows for cleavage of the linker by a protease, thereby facilitating release of the drug from the antibody drug conjugate upon exposure to intracellular proteases, such as lysosomal enzymes. Exemplary amino acid units include, but are not limited to. a dipeptide, a tripeptide, a tetrapeptide, and a pentapeptide. Exemplary dipeptides include: valine-citrulline (vc or val-cit), alanine-phenylalanine (af or ala-phe); phenylalanine-lysine (fk or phe-lys): or N-methyl-valine-citrulline (Me-valcit). Exemplary tripeptides include: glycine-valine-citrulline (gly-val-cit) and glycine-glycine-glycine (gly-gly-gly). In some embodiments, a linker comprising vc (valine-citrulline) unit is preferred. Amino acid units can be designed and optimized in their selectivity for enzymatic cleavage by a particular enzyme, for example, a tumor-associated protease, cathepsin B, C and D, or a plasmin protease.

In one aspect, the linker used in the present disclosure is derived from a crosslinking reagent such as MC (6-maleimidocaproyl), Val-Cit (valine-citrulline), PABC (para-amino-benzyloxycarbonyl), DMEA (dimethylethylamine), Val-Cit-PABC, MC-Val-Cit-PABC, MC-Val-Cit-PABC-DMEA, CL2A, Mal-PEG8-Val-Ala-PABC, GGFG (Glycine-Glycine-Phenylalanine-Glycine), MC-GGFG-aminomethyl, AcBut (4-(4-acetylphenoxy)-butanoic acid), dimethylhydrazide (3-methyl-3-mercaptobutane hydrazide), AcBut-dimethylhydrazide, SPDP (N-succinimidyl-3-(2-pyridyldithio) propionate), SPP (N-succinimidyl 4-(2-pyridyldithio) pentanoate), SPDB (N-succinimidyl 4-(2-pyridyldithio) butanoate), sulfo-SPDB (N-succinimidyl-4-(2-pyridyldithio)-2-sulfo-butanoate), SIA (N-succinimidyl iodoacetate), SIAB (N-succinimidyl (4-iodoacetyl)aminobenzoate), maleimide PEG NHS, SMCC (N-succinimidyl 4 -(maleimidomethyl)cyclohexanecarboxylate), sulfo-SMCC (N-sulfosuccinimidyl 4-(maleimidomethyl)cyclohexanecarboxylate) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

In another aspect, the linker used in the present disclosure is derived from a cross-linking agent such as sulfo-SPDB (N-succinimidyl-4-(2-pyridyldithio)-2-sulfo-butanoate), mc (6-maleimidocaproyl), Val-Cit (valine-citrulline), PABC (para-amino benzyl alcohol), Val-Cit-PABC, mc-Val-Cit-PABC, CL2A, mal-PEG8-Val-Ala-PABC, GGFG (Glycine-Glycine-Phenylalanine-Glycine), mc-GGFG-aminomethyl, AcBut (4-(4-acetylphenoxy)-butanoic acid), dimethylhydrazide (3-methyl-3-mercaptobutane hydrazide), AcBut-dimethylhydrazide or SMCC (N-Succinimidyl 4-(maleimidomethyl)cyclohexane-carboxylate).

In preferred embodiments, the linker is selected from the group consisting of MC (6-maleimidocaproyl), Val-Cit (valine-citrulline), PABC (para-amino-benzyloxycarbonyl), DMEA (dimethylethylamine), Val-Cit-PABC, MC-Val-Cit-PABC, MC-Val-Cit-PABC-DMEA, GGFG (Glycine-Glycine-Phenylalanine-Glycine), MC-GGFG-aminomethyl, AcBut (4-(4-acetylphenoxy)-butanoic acid), and AcBut-dimethylhydrazide. In a more preferred embodiment, the linker is MC-Val-Cit-PABC.

### Pharmaceutical Compositions

In the seventh aspect, the invention provides a pharmaceutical composition comprising (i) the antibody or the antigen binding fragment thereof of the first aspect of the invention, or the bi-specific antibody of the second aspect of the invention, or the nucleic acid of the third aspect of the invention, or the vector of the fourth aspect of the invention, or the host cell of the fifth aspect of the invention, or the ADC of the sixth aspect of the invention; and optionally (ii) a pharmaceutically acceptable carrier or excipient.

The invention provides pharmaceutical composition comprising an antibody of the invention. In some embodiments, the pharmaceutical composition further comprises a pharmaceutically acceptable carrier. The term “pharmaceutically acceptable carrier” includes any and all solvents, buffers, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion). For example, in some embodiments, a composition for intravenous administration typically is a solution in sterile isotonic aqueous buffer.

The antibodies or agents of the invention (also referred to herein as “active compounds”), and derivatives, fragments, analogs and homologs thereof, can be incorporated into pharmaceutical compositions suitable for administration. Such compositions typically comprise the antibody or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention provides therapeutic compositions comprising the anti-CEACAM5/6 dual-binding antibodies or antigen-binding fragments thereof of the present invention. Therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

### Methods of Production

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103) Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies are antibody molecules in which the entire sequence of both the light chain and the heavy chain, including the CDRs, arise from human genes. Such antibodies are termed “humanized antibodies”, “human antibodies”, or “fully human antibodies” herein. Human monoclonal antibodies can be prepared by using trioma technique; the human B-cell hybridoma technique (see Kozbor, et al., 1983 Immunol Today 4:72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80:2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, humanized antibodies can be produced in transgenic plants, as an inexpensive production alternative to existing mammalian systems. For example, the transgenic plant may be a tobacco plant, i.e., Nicotiania benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can be readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the present invention further provides any cell or plant comprising a vector that encodes the antibody of the present invention, or produces the antibody of the present invention.

In addition, an (human) antibody of interest may be produced in fungi. For example, the fungus may be Myceliophthora thermophila (e.g. Myceliophthora thermophila strain C1; Visser et al. (2011) Industrial Biotechnology 7 (3): 214-223). Other examples include Aspergillus species (e.g. A. oryzae (Huynh et al. (2020) Fungal Biology and Biotechnology 7:7), A. niger (Ward et al. (2004) Environ. Microbiol. 70:2567-76), or A. awamori (Joosten et al. (2003) Microb. Cell Fact 2:1)) and Trichoderma species (e.g. T. reesei (Nyyssönen et al. (1993) Biotechnology 11:591-595)). In other instances, the fungus may be a yeast, such as Saccharomyces cerevisiae, Candida boidinii, Hansenula polymorpha, Pichia methanolica, Pichia pastoris, Yarrowia lipolytica, Kluyveromyces lactis or Ogataea minuta (Joosten et al. (2003); Suzuki et al. (2017) J Biosci Bioeng. 124:156-63).

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in WO 2006/008548, WO 2007/096779, WO 2010/109165, WO 2010/070263, WO 2014/141189 and WO 2014/141192.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, are disclosed in PCT publication WO 99/53049.

The antibody can be expressed by a vector containing a DNA segment encoding the single chain antibody described above.

These can include vectors, liposomes, naked DNA, adjuvant-assisted DNA, gene gun, catheters, etc. Vectors include chemical conjugates such as described in WO 93/64701, which has targeting moiety (e.g., a ligand to a cellular surface receptor), and a nucleic acid binding moiety (e.g., polylysine), viral vector (e.g., a DNA or RNA viral vector), fusion proteins such as described in PCT/US 95/02140 (WO 95/22618) which is a fusion protein containing a target moiety (e.g., an antibody specific for a target cell) and a nucleic acid binding moiety (e.g., a protamine), plasmids, phage, etc. The vectors can be chromosomal, non-chromosomal or synthetic.

Preferred vectors include viral vectors, fusion proteins and chemical conjugates. Retroviral vectors include Moloney murine leukemia viruses. DNA viral vectors are preferred. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cell cytoplasm. Avipox virus vectors result in only a short-term expression of the nucleic acid. Adenovirus vectors, adeno-associated virus vectors and herpes simplex virus (HSV) vectors are preferred for introducing the nucleic acid into neural cells. The adenovirus vector results in a shorter-term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include e.g., naked DNA, CaPO4 precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g., adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

### Therapeutic Methods

The antibodies provide herein can be administered to slow or inhibit the progression of a cancer associated with the expression of CEACAM5 and/or CEACAM6. In these applications, a therapeutically effective amount of a composition is administered to a subject in an amount sufficient to inhibit growth, replication or metastasis of cancer cells, or to inhibit a sign or a symptom of the cancer. Suitable subjects may include those diagnosed with a cancer that expresses CEACAM5 and/or CEACAM6, such as small intestine cancer, colorectal cancer, stomach cancer, lung cancer, cervical cancer, pancreatic cancer, esophagus cancer, ovarian cancer, thyroid cancer, bladder cancer, endometrial cancer, breast cancer, liver cancer, prostatic cancer and skin cancer.

Provided herein is a method of treating a cancer associated with the expression of CEACAM5 and/or CEACAM6 in a subject by administering to the subject a therapeutically effective amount of an antibody described herein. In some embodiments, the cancer is small intestine cancer, colorectal cancer, stomach cancer, lung cancer, cervical cancer, pancreatic cancer, esophagus cancer, ovarian cancer, thyroid cancer, bladder cancer, endometrial cancer, breast cancer, liver cancer, prostatic cancer and skin cancer.

Administration of an antibody disclosed herein can also be accompanied by administration of other anti-cancer agents or therapeutic treatments (such as surgical resection of a tumor). Any suitable anti-cancer agent can be administered in combination with the antibodies disclosed herein.

Exemplary anti-cancer agents include, but are not limited to, chemotherapeutic agents, such as, for example, mitotic inhibitors, alkylating agents, antimetabolites, intercalating antibiotics, growth factor inhibitors, cell cycle inhibitors, enzymes, topoisomerase inhibitors, anti-survival agents, biological response modifiers, anti-hormones (e.g., anti-androgens) and anti-angiogenesis agents.

Other anti-cancer treatments include radiation therapy and other antibodies that specifically target cancer cells.

Another common treatment for some types of cancer is surgical treatment, for example surgical resection of a metastatic tumor. Another example of a treatment is radiotherapy, for example administration of radioactive material or energy (such as external beam therapy) to the tumor site to help eradicate the tumor or shrink it prior to surgical resection.

### Methods for Diagnosis and Detection

Methods are provided herein for detecting CEACAM5 and/or CEACAM6 protein in vitro or in vivo. In some cases, CEACAM5 and/or CEACAM6 expression is detected in a biological sample. The sample can be any sample, including, but not limited to, blood samples, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. A biological sample is typically obtained from a mammal, such as a human or non-human primate. In a preferred embodiment, the method is used for a non-diagnostic purpose.

Provided herein is a method of determining if a subject has a cancer associated with the expression of CEACAM5 and/or CEACAM6 by contacting a sample from the subject with an anti-CEACAM5/6 dual-binding antibody disclosed herein; and detecting binding of the antibody to the sample. An increase in binding of the antibody to the sample as compared to binding of the antibody to a control sample identifies the subject as having the cancer.

In another embodiment, provided is a method of confirming a diagnosis of a cancer associated with the expression of CEACAM5 and/or CEACAM6 in a subject by contacting a sample from a subject diagnosed with a cancer associated with the expression of CEACAM5 and/or CEACAM6 with an anti-CEACAM5/6 dual-binding antibody disclosed herein; and detecting binding of the antibody to the sample. An increase in binding of the antibody to the sample as compared to binding of the antibody to a control sample confirms the diagnosis of the cancer in the subject.

In some examples of the disclosed methods, the monoclonal antibody is directly labeled.

In other examples, the methods further include contacting a second antibody that specifically binds the monoclonal antibody with the sample; and detecting the binding of the second antibody. An increase in binding of the second antibody to the sample as compared to binding of the second antibody to a control sample detects a cancer associated with the expression of CEACAM5 and/or CEACAM6 in the subject or confirms the diagnosis of a cancer associated with the expression of CEACAM5 and/or CEACAM6 in the subject.

In some examples, the cancer is small intestine cancer, colorectal cancer, stomach cancer, lung cancer, cervical cancer, pancreatic cancer, esophagus cancer, ovarian cancer, thyroid cancer, bladder cancer, endometrial cancer, breast cancer, liver cancer, prostatic cancer and skin cancer.

In some examples, the control sample is a sample from a subject without cancer. In particular examples, the sample is a blood or tissue sample.

In some embodiments of the methods of diagnosis and detection, the anti-CEACAM5/6 dual-binding antibody is directly labeled with a detectable label. In another embodiment, the anti-CEACAM5/6 dual-binding antibody (the first antibody) is unlabeled and a second antibody or other molecule that can bind the first is labeled. As is well known to one of skill in the art, a secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

In an alternative embodiment, CEACAM5 and/or CEACAM6 can be assayed in a biological sample by a competition immunoassay utilizing CEACAM5 and/or CEACAM6 protein standards labeled with a detectable substance and an unlabeled anti-CEACAM5/6 dual-binding antibody. In this assay, the biological sample, the labeled CEACAM5 and/or CEACAM6 protein standards and the anti-CEACAM5/6 dual-binding antibody are combined and the amount of labeled CEACAM5 and/or CEACAM6 protein standard bound to the unlabeled antibody is determined. The amount of CEACAM5 and/or CEACAM6 in the biological sample is inversely proportional to the amount of labeled CEACAM5 and/or CEACAM6 protein standard bound to the anti-CEACAM5/6 dual-binding antibody.

The immunoassays and methods disclosed herein can be used for a number of purposes. In one embodiment, the anti-CEACAM5/6 dual-binding antibody may be used to detect the production of CEACAM5 and/or CEACAM6 in cells in cell culture. In another embodiment, the antibody can be used to detect the amount of CEACAM5 and/or CEACAM6 in a biological sample, such as a tissue sample, or a blood or serum sample. In some examples, the CEACAM5 and/or CEACAM6 are cell-surface CEACAM5 and/or CEACAM6. In other examples, the CEACAM5 and/or CEACAM6 protein is soluble (e.g. in a cell culture supernatant or in a body fluid sample, such as a blood or serum sample).

In one embodiment, a kit is provided for detecting CEACAM5 and/or CEACAM6 in a biological sample, such as a blood sample or tissue sample. For example, to confirm a cancer diagnosis in a subject, a biopsy can be performed to obtain a tissue sample for histological examination. Kits for detecting a polypeptide will typically comprise a monoclonal anti-CEACAM5/6 dual-binding antibody, such as any of the monoclonal antibodies disclosed herein. In a further embodiment, the antibody is labeled (for example, with a fluorescent, radioactive, or an enzymatic label).

In one embodiment, a kit includes instructional materials disclosing means of use of an anti-CEACAM5/6 dual-binding antibody. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting CEACAM5 and/or CEACAM6 in a biological sample generally includes the steps of contacting the biological sample with an anti-CEACAM5/6 dual-binding antibody. The antibody is allowed to specifically bind under immunologically reactive conditions to form an immune complex, and the presence of the immune complex (bound antibody) is detected directly or indirectly.

The antibodies disclosed herein can also be utilized in immunoassays, such as, but not limited to radioimmunoassays (RIAs), ELISA, or immunohistochemical assays. The antibodies can also be used for fluorescence activated cell sorting (FACS). FACS employs a plurality of color channels, low angle and obtuse light-scattering detection channels, and impedance channels, among other more sophisticated levels of detection, to separate or sort cells (see U.S. Pat. No. 5,061,620). Any of the monoclonal antibodies that bind CEACAM5 and/or CEACAM6, as disclosed herein, can be used in these assays. Thus, the antibodies can be used in a conventional immunoassay, including, without limitation, an ELISA, a RIA, FACS, tissue immunohistochemistry, Western blot or immunoprecipitation.

### EXAMPLES

The present invention is further illustrated by the following examples, which are not intended to limit the present invention. Experimental procedures without specified conditions in the following examples are performed in accordance with conventional procedures and conditions, or in accordance with instructions.

### Example 1. Immunization Strategies and Single B-Cell Cloning Screening of Anti-CEACAM5/6 Dual-Binding Antibodies

### Immunization

Recombinant human CEACAM5 (Uniprot P06731) Fc-tag protein, human CEACAM5 A3B3 His-tag fragment or CHO-K1-huCEACAM5 over expression cell line was used as immunogens to generate anti-CEACAM5 antibodies in immunization using Harbour H2L2 mice (WO2010/070263 A1). The immunization plans are listed in Table 4.

TABLE 4

| | | Animal | | | |
|---|---|---|---|---|---|
| | | Group | | | |
| Group | Immunogen | Size | Strain | Route | Adjuvant |
| 1 | huCEACAM5- | 10 | H2L2 | i.p. | CFA/Ribi/Ribi/ |
| | Fc | | (V2.1) | (250 ul) | Ribi/Ribi |
| 2 | CHO-K1- | 10 | H2L2 | i.p. | NA |
| | huCEACAM5 | | (V2.1) | (250 ul) | |

For protein immunization, each mouse was administrated with 50 μg of the protein for the first boost and 25 μg for following boosts via s.c. and i.p. together with adjuvant (Sigma, S6322). Such immunization was conducted bi-weekly for 5 times. For cell immunization, each mouse was administrated with 1×10⁷ cells in PBS for 5 times on a three-week interval. Final immunization was conducted with immunogen diluted in PBS via i.p. Serum titers were tested against recombinant human CEACAM5 His-tag, recombinant cyno CEACAM5 (XP_005589491.1) His-tag and recombinant human CEACAM1 (Uniprot P13688) His-tag protein (in-house) using ELISA and FACS. Mice with high serum titer against human CEACAM5 and cyno CEACAM5, while with lower titer against human CEACAM1 were selected for subsequent single B-cell cloning Screening.

### Single B-Cell Cloning Screening

### Single B Cell Screening Based on Beacon® Optofluidic System

The Beacon® Optofluidic system uses optical-electric positioning (OEP™) technology to move individual cells. The Beacon optical-electric system is an automated biological instrument and equipment that can allow simultaneous biological function tests, experimental analysis, positive clone selection and other operations under cell culture conditions. The Beacon platform can perform these tasks in a massively parallel, automated manner on thousands of cells.

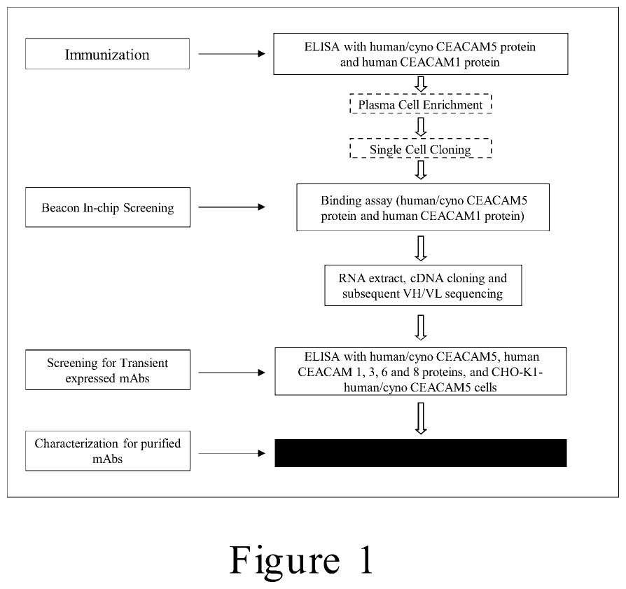

In this application a plasma cell discovery workflow was used. In each experiment, up to 14 k individual plasma cells can be screened for selection of antigen-specific antibodies secreted by positive plasma cells. Then, these plasma cells that secrete specific antibodies were exported to 96-well plates containing cell lysates for subsequent single B cell sequencing to identify the heavy chain and light chain of the antibody produced by a single B cell (monoclonal). The whole screening strategy and progress were represented in FIG. 1.

### Single B Cell Sequencing

The single B cell sequencing method was used to recover antibody heavy chain and light chain sequences from a single plasma cell. Single B cell sequencing has become a powerful tool for obtaining antibody sequences. General procedures include purification of RNA from single plasma cell lysate, reverse transcription synthesis of cDNA, amplification and purification of cDNA, amplification of heavy and light chains, cloning and transfection, and Sanger sequencing. The obtained sequences were subjected to uniqueness and cluster analysis on followed by synthesis of the paired heavy and light chain DNA sequences.

### Example 2. Production and Purification of Anti-CEACAM5/6 Dual-Binding Antibodies

The recombinant plasmids encoding target antibodies were transiently co-transfected into HEK293-6E or 293-F cell cultures using PEI (Polyscience, 24885). After transfection, the cells were incubated at 37° C. with 5% CO₂ and shaking at 120 rpm. The cell culture supernatants collected on day 6-7 days were used for purification. Monoclonal antibodies were purified using Protein A magnetic beads (AmMag Protein A Magnetic Beads, Genscript, L00695).

Antibody purity was examined by SEC-HPLC (Agilent 1260 Infinity II HPLC with Welch Xtimate SEC-300 column, 1×PBS pH 7.4 as mobile phase) and SDS-PAGE (SurePAGE, Bis-Tris, 10×8, 4-12%, 12 wells, Genscript, M00653). 4 recombinant antibodies, termed as PR304169, PR304171, PR304294 and PR304713 were successfully expressed and purified for characterization. The amino acid sequences of the 4 antibodies were listed in Table 1.

In the meantime, an anti-CEACAM5 antibody, Tusamitamab (SAR408377), was also produced following the procedures showed above with sequence information from SAR408377, Proposed INN List 123 Vol. 34, No. 2, 2020. Additionally, an anti-CEACAM6 antibody, Tinurilimab (BAY-1834942) were also produced following the procedures showed above with sequence information from Proposed INN List 83 Vol. 34, No. 1, 2020. These antibodies were used as control and were assigned a code of PR003561 (for Tusamitamab) and PR303563 (for Tinurilimab) respectively. The amino acid sequences of these antibodies were listed in Table 2.

### Example 3. Characterization of Binding Properties of Anti-CEACAM5/6 Dual-Binding Monoclonal Antibodies

### Binding to Human and Cynomolgus CEACAM5 and Human CEACAM 6, 1, 3 and 8 Proteins

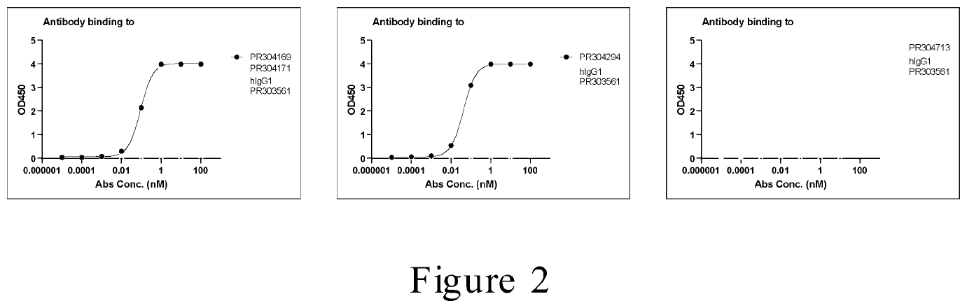

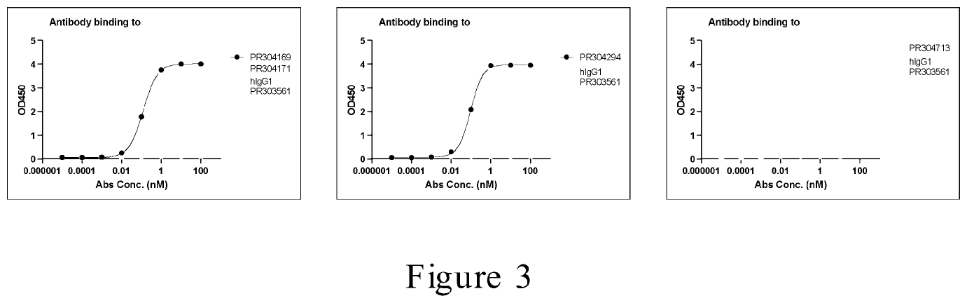

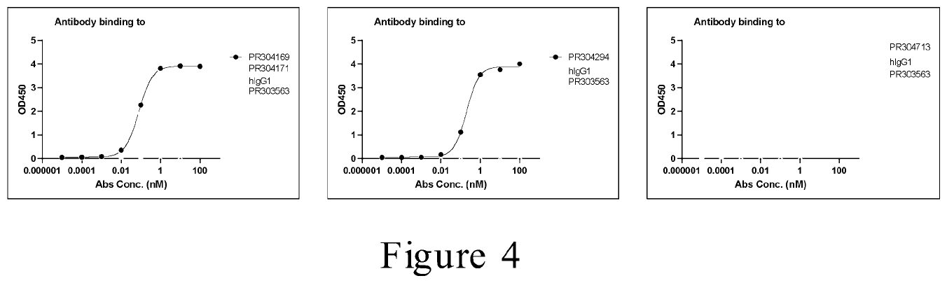

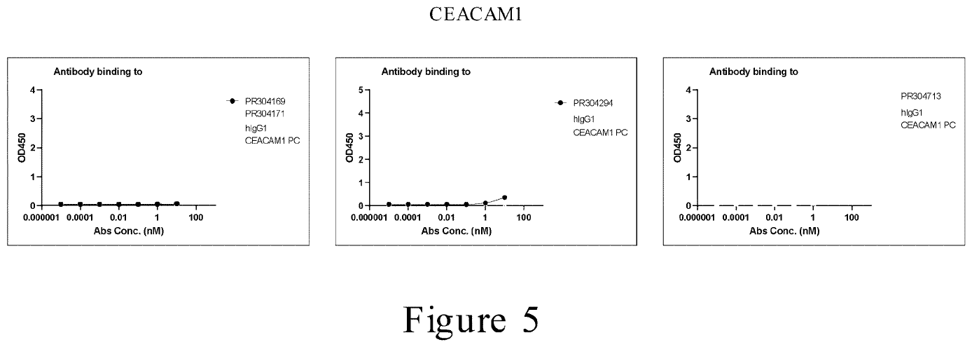

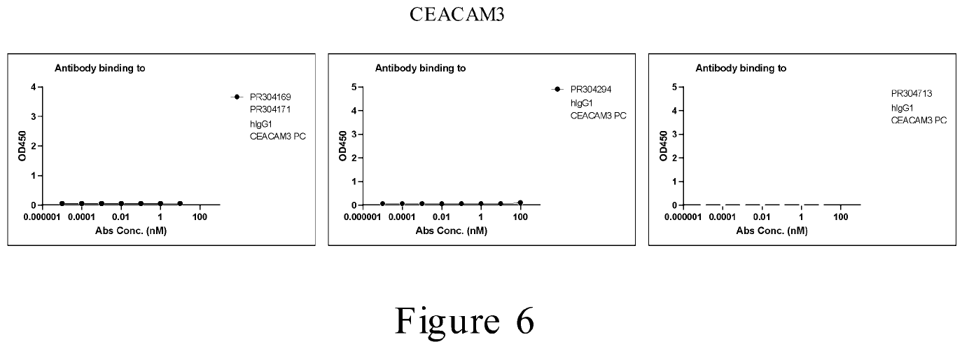

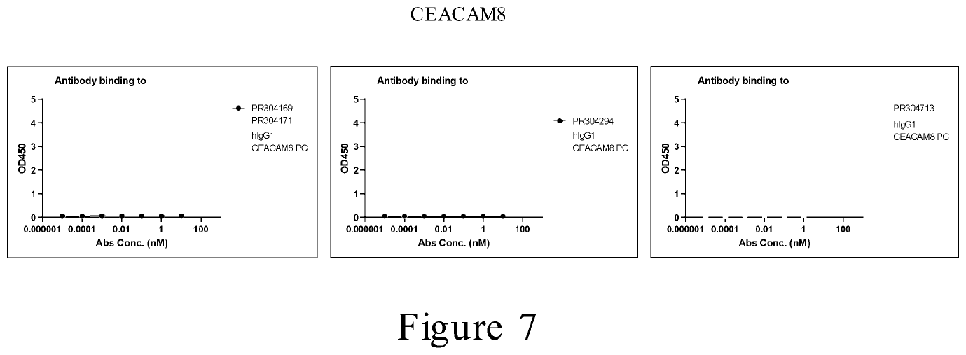

Binding of recombinant anti-CEACAM5/6 dual-binding monoclonal antibodies against human and cynomolgus CEACAM5, and human CEACAM6 (Uniprot P40199), human CEACAM 1, 3 (NP_001806.2, Sino Biological 11933-H08H) and 8 (NP_001807.2, Acro Bio CE8-H5224) were detected by ELISA. Anti-CEACAM5/6 dual-antibodies were serially diluted in staining buffer (PBS containing 2% FBS). Anti-CEACAM5 antibody PR003561 (Tusamitamab), anti-CEACAM6 antibody PR303563 (Tinurilimab), anti-CEACAM1 antibody CEACAM1PC (SinoBiological, Cat: 10822-MM02, Mouse Monoclonal), anti-CEACAM3 antibody CEACAM3PC (SinoBiological, Cat: 11933-MM03, Mouse Monoclonal) and anti-CEACAM8 antibody CEACAM8PC (SinoBiological, Cat: 11729-MM04, Mouse Monoclonal) were used as control. ELISA was used to test the binding between antibody containing supernatant or purified antibodies and the antigen. Briefly, the antigens were coated on a 96-well high binding plate overnight at 4° C. The plate was washed with PBST three times, incubated with blocking buffer (2% Bovine Serum Albumin) for one hour at 37° C., washed with PBST three times and incubated with antibody containing supernatant or purified antibodies at desired dilutions or concentrations for one hour at 37° C. A secondary antibody, anti-human Fc-HRP (Jackson Laboratory, 109-035-097) was used for detection of the targeted antibody. TMB and stopping solution were used for detection of HRP signal. The HRP signal was read on a SPECTRAMAX plate reader. EC50 was determined from the ELISA test result (see Tables 5 and FIGS. 2-7).

As shown in Table 5 and FIGS. 2-7, four antibodies PR304169, PR304171, PR304713 and PR304294, showed good binding activities to both human and cyno CEACAM5 proteins, but no binding to CEACAM1, CEACAM3 and CEACAM8. A remarkable property is that all four antibodies also show good binding activity to human CEACAM6, while the control antibody PR303561 (Tusamitamab) does not cross react to CEACAM6. This could potentially transfer to larger coverage when applied to therapeutics. No such antibody has been reported so far. This binding property provides broader coverage of cancer cells.

TABLE 5
ELISA binding of anti-CEACAM5/6 dual-binding antibodies to various CEACAM proteins

| | ELISA | | | | | |
|---|---|---|---|---|---|---|
| | EC50 (nM) | | | | | |
| | | cyno | | | | |
| Abs | huCEACAM5 | CEACAM5 | huCEACAM6 | huCEACAM1 | huCEACAM3 | huCEACAM8 |
| PR304169 | 0.09 | 0.12 | 0.077 | No binding | No binding | No binding |
| PR304171 | 0.15 | 1.25 | 24.13 | No binding | No binding | No binding |
| PR304713 | 0.01 | 0.021 | 0.004 | No binding | No binding | No binding |
| PR304294 | 0.04 | 0.09 | 0.22 | No binding | No binding | No binding |
| PR303561 | 0.02 | 0.01 | No binding | No binding | No binding | No binding |
| PR303563 | No binding | No binding | 0.01 | No binding | No binding | No binding |

### Binding to Human or Cynomolgus CEACAM5/6 Stable Cell Lines

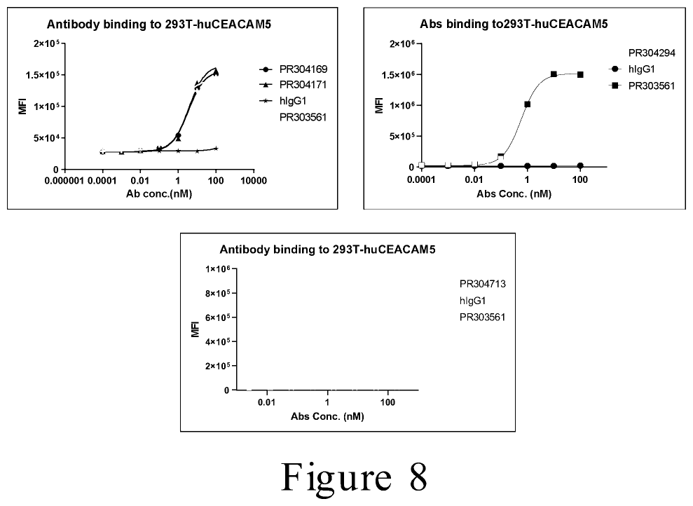

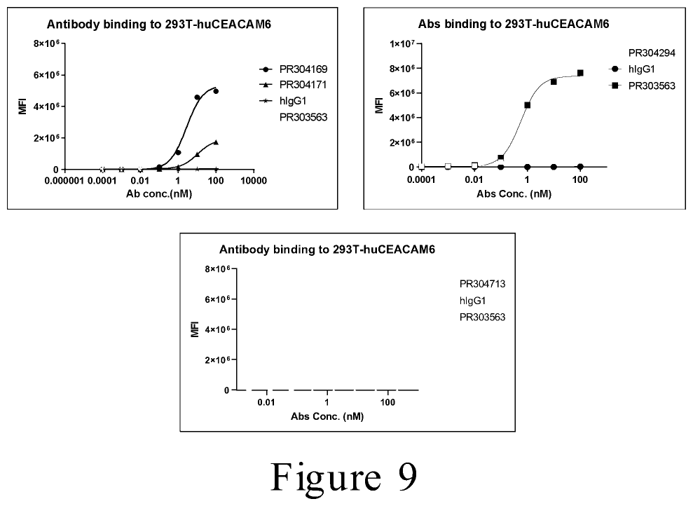

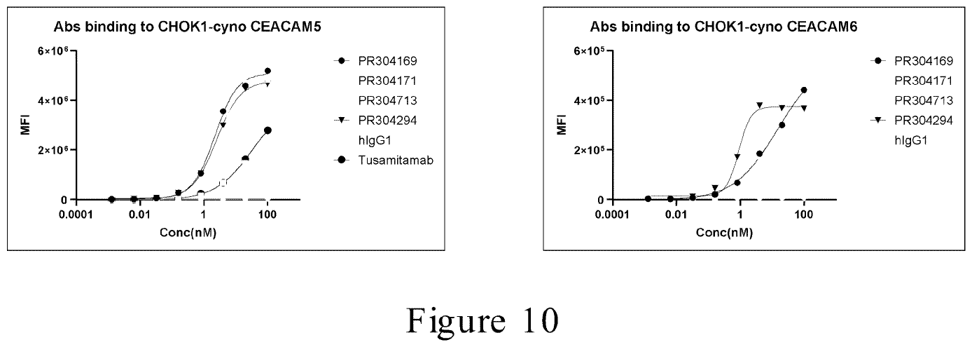

Binding of recombinant anti-CEACAM5/6 dual-binding antibodies against human or cynomolgus CEACAM5 or CEACAM6 stable cells were detected by flow cytometry. The stable cell lines include 293T cell lines that had been transfected to overexpress human CEACAM5 (293T-huCEACAM5 cells) or CEACAM6 (293T-huCEACAM6 cells) on the surface and CHO-K1 cell lines that had been transfected to overexpress cynomolgus CEACAM5 (CHOK1-cyno CEACAM5 cells) or CEACAM6 (CHOK1-cyno CEACAM6 cells) on the surface. Anti-CEACAM5/6 dual-binding antibodies were incubated with the stable cell lines. Anti-CEACAM5/6 dual-binding antibodies were serially diluted in staining buffer (PBS containing 2% FBS). 50 μL of diluted antibody solution was added to 50 μL of cell suspension containing 1-2×10⁵ cells and incubated at 4° C. for 1 hour. The cells were washed twice with staining buffer and added with 100 μL/well of 1:1000 diluted florescent labeled anti-human IgG antibody (Alexa Fluor® 488 AffiniPure Goat Anti-Human IgG (H+L), Jackson Cat 109-545-088). After 1-hour incubation at 4° C., cells were washed twice and run on a flow cytometer. PR303561 and non-relevant IgG isotype control (Crownbio, hIgG1) were used as positive control and negative control respectively. The results were shown in FIGS. 8-10 and Tables 6 below.

The results indicated that the anti-CEACAM5/6 dual-binding antibodies have very good binding activity against both human and cynomolgus CEACAM5 and CEACAM6 on cell membrane.

TABLE 6
Anti-CEACAM5/6 dual-binding antibodies binding EC50s
to cell surface-bound CEACAM5/6 detected by FACS

| | FACS Binding | | | | | | |
|---|---|---|---|---|---|---|---|
| | EC50 (nM) | | | | | | |
| | 293T- | 293T- | CHOK1- | CHOK1- | | | |
| Abs | huCEACAM5 | huCEACAM6 | cynoCECAM5 | cynoCECAM6 | LS 174T | BxPC3 | A549 |
| PR304169 | 3.39 | 2.74 | 2.20 | 15.04 | 13.64 | 16.1 | 8.57 |
| PR304171 | 3.64 | 10.59 | 14.88 | 4.58 | 8.92 | 45.3 | ND |
| PR304713 | 6.44 | 3.43 | 3.19 | 9.50 | 10.75 | 12.92 | 9.02 |
| PR304294 | 1.26 | 1.325 | 2.51 | 0.88 | 3.87 | 6.98 | 1.49 |
| PR303561 | 1.04 | No binding | 33.40 | No binding | 1.74 | 3.06 | No binding |

### Binding to CEACAM5 and CEACAM6 Expressing Endogenous Cell Lines LS 174T and BxPC3

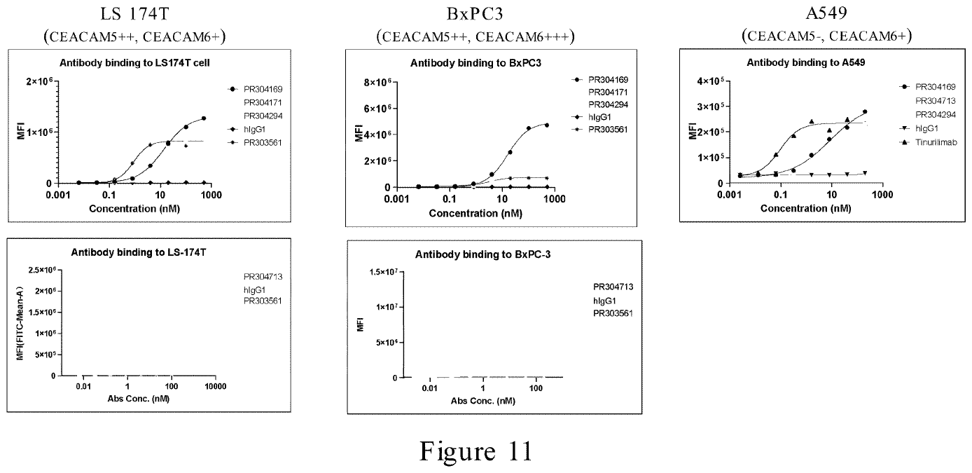

Similar procedure was followed as described above only replace the stable cell lines to LS 174T (ATCC CL-188), BxPC3 (ATCC CRL-1687) and A549 cell lines, which simultaneously express different levels of membrane-bound endogenous CEACAM5 and CEACAM6. The binding results showed in FIGS. 11 and Tables 6 above.

FIG. 11 and Table 6 showed good binding ability of the anti-CEACAM5/6 dual-binding antibodies to membrane-bound endogenous human CEACAM5 and CEACAM6 on LS 174T, BxPC3 and A549 cell lines. The dual-binding antibodies showed much larger saturated binding capacity than the control antibodies PR303561.

### Example 4. Determination of Binding Affinity of Anti-CEACAM5/6 Dual-Binding Antibodies to Soluble CEACAM5 and CEACAM6 by Octet

Binding kinetics of recombinant anti-CEACAM5/6 antibodies to CEACAM5 and CEACAM6 were analyzed by Octet. In Octet analysis, recombinant human CEACAM5 Fc-tag protein (#Uniprot P06731) or CEACAM6 Fc-tag protein (Uniprot P40199) was serially diluted with 1×kinetic buffer (Fortebio). Anti-CEACAM5/6 dual-binding antibodies were diluted to 5 μg/mL. Then diluted antibodies, antigen and regeneration buffer (10 mM glycine pH 1.75) were added to 96-well plates (Greiner). Rate constants for association and dissociation were measured using AHC sensor (Fortebio). The sensor surface was regenerated after each binding experiment with regeneration buffer. The traces were processed using ForteBio Data Analysis Software (version 8.0, Pall ForteBio, CA, USA).

KD values of anti-CEACAM5/6 dual-binding antibodies were summarized in Table 7.

TABLE 7
Antibody KD values detected by Octet

Octet Affinity
K_{D} (M)

| Abs | huCEACAM5 | huCEACAM6 |
|---|---|---|
| PR304169 | 1.69E−09 | 3.49E−09 |
| PR304713 | 2.50E−09 | 7.70E−09 |
| PR304294 | 5.48E−09 | 1.99E−08 |
| PR303561 | 6.00E−09 | No binding |

### Example 5. Epitope Binning of Anti-CEACAM5/6 Dual-Binding Antibodies by Octet

In epitope binning analysis using Octet, antibodies were tested against other antibodies in a pairwise fashion to see if antibodies block one another's binding to the epitope of the antigen. The first and second antibodies was diluted into same concentration which could saturate the binding of immobilized antigens. Recombinant CEACAM5-his protein was biotinylationed and was then used as antigen and immobilized to SA sensor. The first antibody was associated with antigen and then followed by the second antibody. Data were analyzed by ForteBio Data Analysis Software (version 8.0, Pall ForteBio). The rate of RU values of the same antibody as 2^{nd} antibody to as 1^{st} antibody was calculated as the co-binding rate of this antibody to other antibodies. Generally speaking, a rate of >50% and near 100% indicates the two antibodies bind to different epitope bins; a rate of <20% indicates an overlapping bin of two assessed antibodies. The co-binding rates were showed in Table 8.

As shown in Table 8, all anti-CEACAM5/6 dual-binding antibodies belongs to the same epitope bin, which is different from that of the control antibody PR303561.

TABLE 8
Epitope binning results of anti-CEACAM5/6
dual-binding antibodies determined by Octet

| Abs | PR304169 | PR304171 | PR304294 | PR303561 |
|---|---|---|---|---|
| PR304169 | −2.50% | −4.99% | 8.75% | 105.68% |
| PR304171 | 20.41% | 13.71% | 24.37% | 101.26% |
| PR304294 | 1.02% | −4.88% | 1.80% | 101.93% |
| PR303561 | 109.51% | 101.15% | 98.53% | 8.80% |

### Example 6. Soluble CEACAM5 or CEACAM6 Interference Assay of the Anti-CEACAM5/6 Dual-Binding Antibodies

These the anti-CEACAM5/6 dual-binding antibodies were then tested for their bindings to 293T-huCEACAM5, 293T-huCEACAM6, LS 174T, and BxPC3 cells expressing CEACAM5 and CEACAM6 in the presence or absence of 100 nM soluble CEACAM5 or CEACAM6. The experiments were performed following the similar procedure showed in Example 3 (Binding to CEACAM5 and CEACAM6 expressing endogenous cell lines LS 174T and BxPC3), except a 100 nM soluble CEACAM5 or CEACAM6 was added to serial diluted antibodies.

The results showed that the binding of these antibodies all decreased when adding 100 nM soluble CEACAM5 or CEACAM6, but mostly the EC50 changes are within 5-fold, indicating minimum interference by soluble CEACAM5 or CEACAM6 to the anti-CEACAM5/6 antibodies. This indicates that these antibodies have good resistance to soluble protein interference.

TABLE 9
Fold changes of EC50 at the presence of 100 nM soluble proteins

| | | | LS174T | BxPC3 |
|---|---|---|---|---|
| | 293T- | 293T- | (CEACAM5++, | (CEACAM5++, |
| | huCEACAM5 | huCEACAM6 | CEACAM6+) | CEACAM6+++) |
| PR304169 + CEACAM5 | 6.8 | | 5.2 | 5.6 |
| PR304169 + CEACAM6 | | 11.1 | 2.8 | 7.7 |
| PR304713 + CEACAM5 | 3.9 | | 5.2 | 4.8 |
| PR304713 + CEACAM6 | | 8.2 | 5.2 | 5.5 |
| PR304294 + CEACAM5 | 4.1 | | 5.4 | 11.0 |
| PR304294 + CEACAM6 | | 2.1 | 1.1 | 1.2 |
| PR303561 + CEACAM5 | 3.9 | | 7.3 | 6.9 |
| PR303563 + CEACAM6 | | 7.3 | 6.9 | 4.1 |

### Example 7. Internalization of Antibodies on 293T-huCEACAM5, 293T-huCEACAM6, LS 174T, HPAC or BxPC3 by pHAb Amine Reactive Dye

The pHAb Amine Reactive Dye (Promega, Cat #G9841) was used to determine the antigen-based internalization of anti-CEACAM5/6 dual-binding antibodies on 293T-huCEACAM5, 293T-huCEACAM6, LS 174T, HPAC or BxPC3 cells.

pHAb Dyes are pH sensor dyes that have very low fluorescence at pH >7 and a dramatic increase in fluorescence as the pH of the solution becomes acidic. In this case, when antibody labeled with pHAb dyes binds outside cells in neutral pH, no or very low fluorescence could be monitored. But after internalization, fluorescence will become stronger in lower pH environments in endosomes and lysosomes.

Antibodies were labeled with pHAb Dyes and calculated for DARs following the kit instructions. The labeled antibodies were incubated with on 293T-huCEACAM5 and LS 174T at 4° C. (the temperature theoretically has very low internalization activities, so will be used as background control) or 37° C. for 6 hours. Then a fluorescence with excitation maxima (Ex) at 532 nm and emission maxima (Em) at 560 nm were detected. The final normalized results will be the fluorescence intensity from 37° C. subtracting the background from 4° C. and them divided by DARs of pHAb Dye of the antibody. The higher value indicates the higher internalization activity.

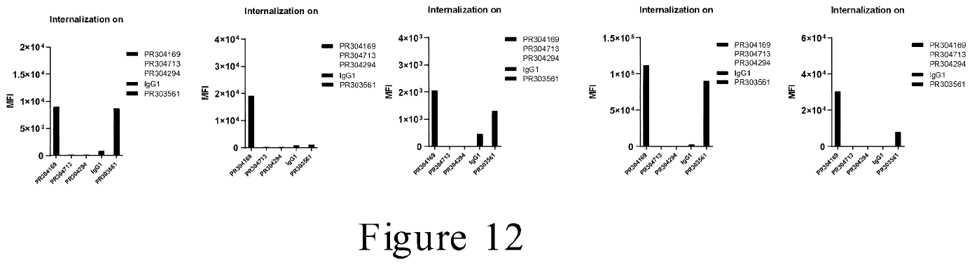

The internalization results were shown in FIGS. 12 and Table 10.

As shown in the FIG. 12, the anti-CEACAM5/6 dual-binding antibodies have good internalization activity. Good internalization will be helpful to improve the efficacy of antibodies used in ADC since most ADCs need internalization to release the toxins, and will also be useful for antibody drugs since it could help to decrease the number of CEACAM5 or CEACAM6 on cell membranes.

TABLE 10
Internalization on 293T-huCEACAM5, 293T-
huCEACAM6, LS174T, HPAC and BxPC3

| | MFI (37° C.-4° C.)/DAR | | | | |
|---|---|---|---|---|---|
| Antibody | 293T- | 293T- | | | |
| Name | huCEACAM5 | huCEACAM6 | LS174T | HPAC | BxPC3 |
| PR304169 | 8987 | 19258 | 2078 | 112658 | 30709 |
| PR304713 | 16613 | 35633 | 3348 | 135448 | 55186 |
| PR304294 | 11260 | 14733 | 2327 | 129768 | 23048 |
| PR303561 | 8722 | 1256 | 1319 | 91001 | 8231 |
| IgG1 | 888 | 975 | 468 | 3014 | 0 |

### Example 8. Internalization of the Anti-CEACAM5/6 Dual-Binding Antibodies on LS 174T, A549, BxPC3 and HPAC with Soluble Protein Interference

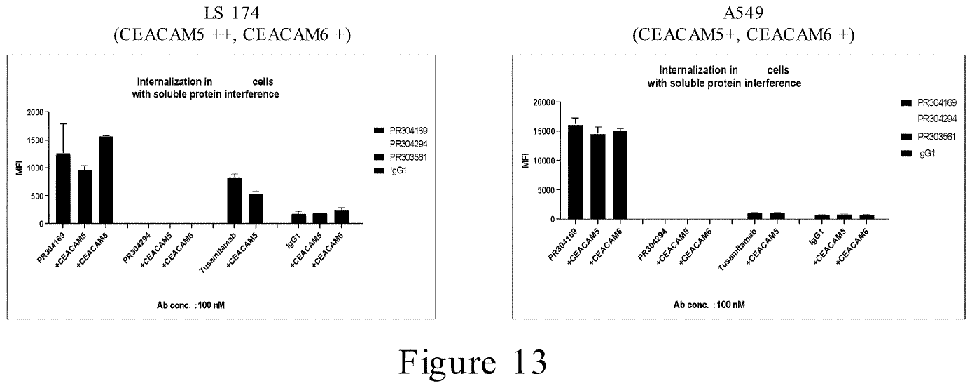

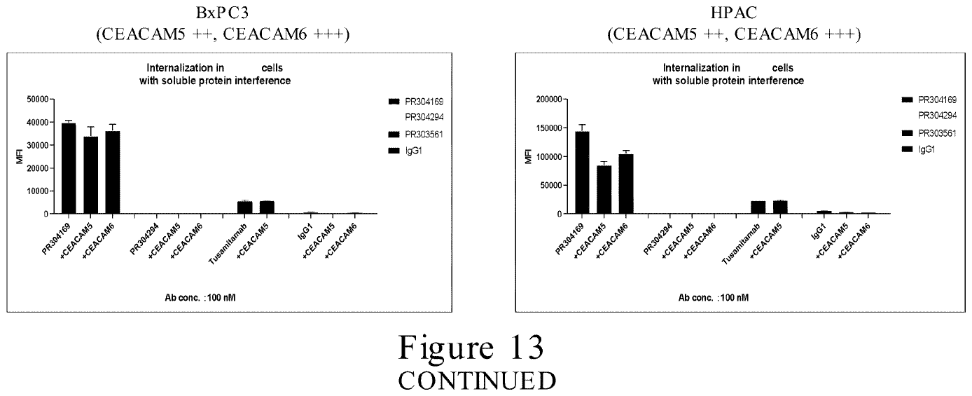

The internalization of the anti-CEACAM5/6 dual-binding antibodies on LS 174T, A549, BxPC3 and HPAC cell lines with soluble protein interference were performed following the similar procedure shown in Example 7, except a 100 nM soluble CEACAM5 or CEACAM6 was added to the system. The experiment results were shown in FIG. 13.

The results showed that there is no interference on the internalization activity of the anti-CEACAM5/6 dual-binding antibodies from soluble CEACAM5 or CEACAM6 proteins.

### Example 9. In Vitro Cytotoxicity of Anti-CEACAM5/6 Antibody-MMAE Conjugated ADCs

In vitro cytotoxicity study was conducted on 293T-huCEACAM5, 293T-huCEACAM6, LS 174T and BxPC3 cells. The anti-CEACAM5/6 dual-binding antibodies were conjugated to MC-Val-Cit-PABC-MMAE to form ADCs at DAR=~4. MC-Val-Cit-PABC-MMAE was purchased commercially (MedChemExpress, HY-15575), and the conjugation was performed as follows. The sample buffer containing the antibodies was adjusted to needed pH by dialysis at 4° C. overnight and adjusted to a concentration of 5 mg/ml. Relative equivalent of TCEP (vs protein) was added to the sample buffer, shaken and incubated at room temperature for 3 hours. Then relative equivalent (vs protein) MC-Val-Cit-PABC-MMAE dissolved in DMSO was added to the reaction sample directly, and incubated at room temperature for 2~6 hours. Then 10 equivalent cysteine (vs protein) was added to the reaction sample to quench the reaction. Excess small molecules were removed by dialysis, and ADCs comprising the anti-CEACAM5/6 antibodies conjugated to MC-Val-Cit-PABC-MMAE were obtained. For the cytotoxicity experiment, cells were incubated with serial diluted ADCs for 6 days. The viability was measured by CellTiter-Glo kit (Promega, G7572). Data was analyzed in GraphPad Prism to acquire the IC50 of different ADCs.

The results in Table 11 indicated that PR304169, PR304713 and PR304294 based ADCs showed comparable or better in vitro cytotoxicity to PR303561 based ADC on CEACAM5 overexpressed 293T cells as well as LS 174T and BxPC3 cells, and also showed very good in vitro cytotoxicity in human CEACAM6 overexpressed 293T cells, indicating broader cell line coverage.

TABLE 11
IC50 of anti-CEACAM5/6 dual binding antibody-
MMAE conjugated ADCs on different cell lines

| | 293T- | 293T- | LS | |
|---|---|---|---|---|
| ADC Name | huCEACAM5 | huCEACAM6 | 174T | BxPC3 |
| PR304169-MMAE | 0.24 | 0.43 | ~35 | 2.38 |
| PR304294-MMAE | 0.032 | 0.46 | ~100 | 0.57 |
| PR304713-MMAE | 0.64 | 0.75 | ~100 | 3.09 |
| PR303561-MMAE | 0.016 | — | ~100 | 1.8 |
| MMAE | 0.001 | 0.005 | 0.5 | 0.03 |
| IgG-MMAE | 66 | 72 | 700 | ~100 |

### Example 10. In Vivo Efficacy of Anti-CEACAM5/6 Antibody-MMAE Conjugated ADCs

For the evaluation of anti-tumor activity of anti-CEACAM5/6 ADCs, in vivo efficacy study was conducted on CDX mouse model with both MKN45 gastric tumor and LS 174T colon cancer tumor cell lines. Tumor volumes were measured twice weekly in two dimensions using a caliper, and the volume was expressed in mm³ using the formula: V=0.5 a×b², where a and b are the long and short diameters of the tumor, respectively. Tumor growth inhibition (TGI): TGI %=(1-Tᵢ/Cᵢ)×100. Tᵢ and Cᵢ are the mean tumor volumes of the treatment and control groups, respectively, on a given day.

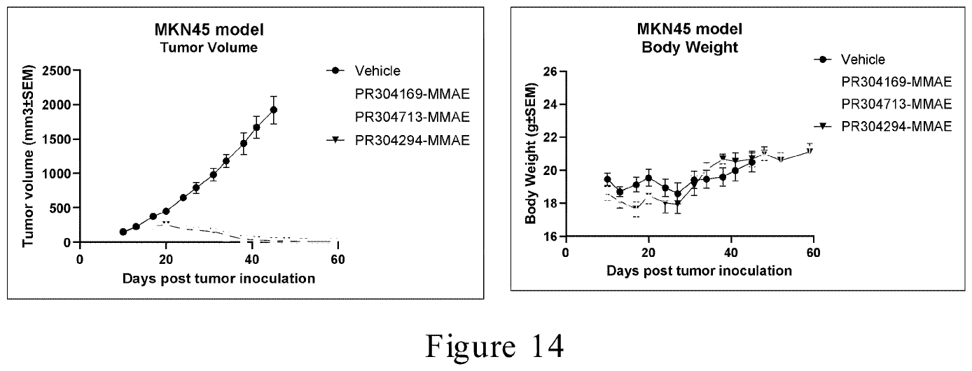

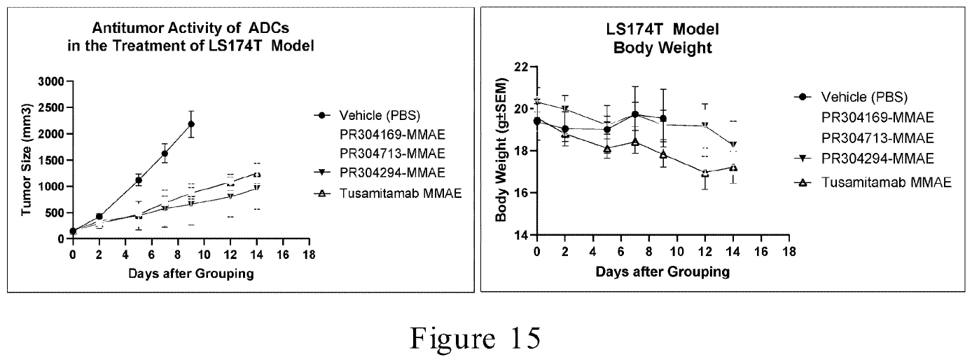

The dosing scheme and results are presented in FIG. 14 (A and B), Table 12, FIG. 15 (A and B) and Table 13. As shown in figures and tables, both PR304169, PR304713 and PR304294 based ADCs showed comparable or better in anti-tumor activity to PR303561 based ADC.

TABLE 12
Evaluation of anti-tumor activity of ADC
against MKN45 gastric cancer tumor

| ADC Name | Dose | TGI (%) on Day 35 |
|---|---|---|
| PR304169-MMAE | 10 mpk, QW × 3 doses | 99.74 |
| PR304713-MMAE | 10 mpk, QW × 3 doses | 96.73 |
| PR304294-MMAE | 10 mpk, QW × 3 doses | 98.75 |

TABLE 13
Evaluation of anti-tumor activity of ADC
against LS 174T colon cancer tumor

| ADC Name | Dose | TGI (%) on Day 9 |
|---|---|---|
| PR304169-MMAE | 10 mpk, QW × 3 doses | 56.82 |
| PR304713-MMAE | 10 mpk, QW × 3 doses | 75.72 |
| PR304294-MMAE | 10 mpk, QW × 3 doses | 70.12 |
| PR303561-MMAE | 10 mpk, QW × 3 doses | 60.67 |

1. An antibody that binds to both CEACAM5 and CEACAM6, or an antigen binding fragment thereof, wherein the antibody comprises a heavy chain variable region (VH) and a light chain variable region (VL), and wherein
(1) the VH comprises the HCDRs 1-3 of a VH having the amino acid sequence set forth in SEQ ID NO: 26, and the VL comprises the LCDRs 1-3 of a VL having the amino acid sequence set forth in SEQ ID NO: 27;
(2) the VH comprises the HCDRs 1-3 of a VH having the amino acid sequence set forth in SEQ ID NO: 7, and the VL comprises the LCDRs 1-3 of a VL having the amino acid sequence set forth in SEQ ID NO: 8;
(3) the VH comprises the HCDRs 1-3 of a VH having the amino acid sequence set forth in SEQ ID NO: 17, and the VL comprises the LCDRs 1-3 of a VL having the amino acid sequence set forth in SEQ ID NO: 18; or
(4) the VH comprises the HCDRs 1-3 of a VH having the amino acid sequence set forth in SEQ ID NO: 31, and the VL comprises the LCDRs 1-3 of a VL having the amino acid sequence set forth in SEQ ID NO: 32.

2. The antibody or antigen binding fragment thereof according to claim 1, wherein
(1) the VH comprises HCDRs 1-3 comprising the amino acid sequences set forth in SEQ ID NOs: 21, 22 and 23 respectively, and the VL comprises LCDRs 1-3 comprising the amino acid sequences set forth in SEQ ID NOs: 24, 5 and 25 respectively;
(2) the VH comprises HCDRs 1-3 comprising the amino acid sequences set forth in SEQ ID NOs: 1, 2 and 3 respectively, and the VL comprises LCDRs 1-3 comprising the amino acid sequences set forth in SEQ ID NOs: 4, 5 and 6 respectively;
(3) the VH comprises HCDRs 1-3 comprising the amino acid sequences set forth in SEQ ID NOs: 11, 12 and 13 respectively, and the VL comprises LCDRs 1-3 comprising the amino acid sequences set forth in SEQ ID NOs: 14, 15 and 16 respectively; or
(4) the VH comprises HCDRs 1-3 comprising the amino acid sequences set forth in SEQ ID NOs: 1, 30 and 3 respectively, and the VL comprises LCDRs 1-3 comprising the amino acid sequences set forth in SEQ ID NOs: 4, 5 and 6 respectively.

3. The antibody or antigen binding fragment thereof according to claim 1 or 2, wherein:
(1) the VH comprises an amino acid sequence having at least 80%, at least 85%, at least 90%, at least 95%, at least 98%, at least 99%, or 100% sequence identity to SEQ ID NO: 26, and the VL comprises an amino acid sequence having at least 80%, at least 85%, at least 90%, at least 95%, at least 98%, at least 99%, or 100% sequence identity to SEQ ID NO: 27;
(2) the VH comprises an amino acid sequence having at least 80%, at least 85%, at least 90%, at least 95%, at least 98%, at least 99%, or 100% sequence identity to SEQ ID NO: 7, and the VL comprises an amino acid sequence having at least 80%, at least 85%, at least 90%, at least 95%, at least 98%, at least 99%, or 100% sequence identity to SEQ ID NO: 8;
(3) the VH comprises an amino acid sequence having at least 80%, at least 85%, at least 90%, at least 95%, at least 98%, at least 99%, or 100% sequence identity to SEQ ID NO: 17, and the VL comprises an amino acid sequence having at least 80%, at least 85%, at least 90%, at least 95%, at least 98%, at least 99%, or 100% sequence identity to SEQ ID NO: 18; or
(4) the VH comprises an amino acid sequence having at least 80%, at least 85%, at least 90%, at least 95%, at least 98%, at least 99%, or 100% sequence identity to SEQ ID NO: 31, and the VL comprises an amino acid sequence having at least 80%, at least 85%, at least 90%, at least 95%, at least 98%, at least 99%, or 100% sequence identity to SEQ ID NO: 32.

4. The antibody or antigen binding fragment thereof according to any one of claims 1-3, wherein the antibody comprises a heavy chain (HC) and a light chain (LC), and wherein:
(1) the HC comprises an amino acid sequence having at least 80%, at least 85%, at least 90%, at least 95%, at least 98%, at least 99%, or 100% sequence identity to SEQ ID NO: 28, and the LC comprises an amino acid sequence having at least 80%, at least 85%, at least 90%, at least 95%, at least 98%, at least 99%, or 100% sequence identity to SEQ ID NO: 29;
(2) the HC comprises an amino acid sequence having at least 80%, at least 85%, at least 90%, at least 95%, at least 98%, at least 99%, or 100% sequence identity to SEQ ID NO: 9, and the LC comprises an amino acid sequence having at least 80%, at least 85%, at least 90%, at least 95%, at least 98%, at least 99%, or 100% sequence identity to SEQ ID NO: 10;
(3) the HC comprises an amino acid sequence having at least 80%, at least 85%, at least 90%, at least 95%, at least 98%, at least 99%, or 100% sequence identity to SEQ ID NO: 19, and the LC comprises an amino acid sequence having at least 80%, at least 85%, at least 90%, at least 95%, at least 98%, at least 99%, or 100% sequence identity to SEQ ID NO: 20; or
(4) the HC comprises an amino acid sequence having at least 80%, at least 85%, at least 90%, at least 95%, at least 98%, at least 99%, or 100% sequence identity to SEQ ID NO: 33, and the LC comprises an amino acid sequence having at least 80%, at least 85%, at least 90%, at least 95%, at least 98%, at least 99%, or 100% sequence identity to SEQ ID NO: 34.

5. The antibody or antigen binding fragment thereof according to any one of claims 1-4, wherein the antibody is a murine antibody, a chimeric antibody, a humanized antibody, or a human antibody.

6. The antibody or the antigen binding fragment thereof according to any one of claims 1-5, wherein the antibody is of an isotype selected from the group consisting of IgG, IgA, IgM, IgE and IgD.

7. The antibody or the antigen binding fragment thereof according to any one of claims 1-6, wherein the antibody is of a subtype selected from the group consisting of IgG1, IgG2, IgG3, and IgG4.

8. The antibody or the antigen binding fragment thereof according to any one of claims 1-7, wherein the antigen binding fragment is selected from the group consisting of Fab, Fab′, F(ab′)₂, Fd, Fd′, Fv, scFv, ds-scFv and dAb.

9. The antibody or the antigen binding fragment thereof according to any one of claims 1-8, wherein the antibody is a monoclonal antibody, a bi-specific or a multi-specific antibody.

10. The antibody or the antigen binding fragment thereof according to any one of claims 1-9, wherein the antibody is monovalent, bivalent or multivalent.

11. The antibody or antigen binding fragment thereof according to any one of claims 1-10, wherein the antibody or antigen binding fragment is attached to a fluorescent label, radiolabel or a drug moiety.

12. The antibody or antigen binding fragment thereof according to claim 11, wherein the antibody or antigen binding fragment is attached to a drug moiety, and the drug moiety is selected from the group consisting of microtubule inhibitors, antibiotics, DNA synthesis inhibitors, topoisomerase inhibitors, RNA polymerase II inhibitors, and RNA spliceosome inhibitors.

13. The antibody or antigen binding fragment thereof according to claim 12, wherein the drug moiety is selected from the group consisting of MMAE, MMAF, duocarmycin, DM1, DM4, SN-38, Dxd, calicheamicin, doxorubicin, and PBDs (Benzodiazepines).

14. The antibody or antigen binding fragment thereof according to claim 11 or 12, wherein the antibody or antigen binding fragment is attached to the drug moiety via a linker.

15. The antibody or antigen binding fragment thereof according to claim 14, wherein the linker comprises a cleavable linker or a non-cleavable linker.

16. The antibody or antigen binding fragment thereof according to claim 15, wherein the cleavable linker is selected from the group consisting of an acid-labile linker, a hydrophilic linker, a protease-sensitive linker, a photolabile linker, a hydrazone linker, a dimethyl linker, and a disulfide-containing linker.

17. The antibody or antigen binding fragment thereof according to claim 15, wherein the linker is selected from the group consisting of MC (6-maleimidocaproyl), Val-Cit (valine-citrulline), PABC (para-amino-benzyloxycarbonyl), DMEA (dimethylethylamine), Val-Cit-PABC, MC-Val-Cit-PABC, MC-Val-Cit-PABC-DMEA, GGFG (Glycine-Glycine-Phenylalanine-Glycine), MC-GGFG-aminomethyl, AcBut (4-(4-acetylphenoxy)-butanoic acid), and AcBut-dimethylhydrazide, preferably MC-Val-Cit-PABC.

18. A bi-specific antibody, comprising the antibody or antigen-binding fragment thereof according to any one of claims 1-10 and a second antigen binding region specifically binding to a tumor associated antigen, an immune cell antigen, or an immune checkpoint molecule.

19. A nucleic acid comprising a nucleotide sequence encoding the antibody or the antigen binding fragment thereof according to any one of claims 1-10 or the bi-specific antibody of claim 18.

20. A vector comprising the nucleic acid according to claim 19.

21. A host cell comprising the nucleic acid according to claim 19 or the vector according to claim 20.

22. An antibody-drug conjugate (ADC), comprising the antibody or the antigen-binding fragment thereof according to any one of claims 1-10 or the bi-specific antibody according to claim 18, and a drug moiety conjugated thereto via a linker.

23. The antibody-drug conjugate according to claim 22, wherein the drug moiety is selected from the group consisting of microtubule inhibitors, antibiotics, DNA synthesis inhibitors, topoisomerase inhibitors, RNA polymerase II inhibitors, and RNA spliceosome inhibitors.

24. The antibody-drug conjugate according to claim 23, wherein the drug moiety is selected from the group consisting of MMAE, MMAF, duocarmycin, DM1, DM4, SN-38, Dxd, calicheamicin, doxorubicin, and PBDs (Benzodiazepines).

25. The antibody-drug conjugate according to any one of claims 22-24, wherein the linker comprises a cleavable linker or a non-cleavable linker.

26. The antibody-drug conjugate according to claim 25, wherein the cleavable linker is selected from the group consisting of an acid-labile linker, a hydrophilic linker, a protease-sensitive linker, a photolabile linker, a hydrazone linker, a dimethyl linker, and a disulfide-containing linker.

27. The antibody-drug conjugate according to claim 25, wherein the linker is selected from the group consisting of MC (6-maleimidocaproyl), Val-Cit (valine-citrulline), PABC (para-amino-benzyloxycarbonyl), DMEA (dimethylethylamine), Val-Cit-PABC, MC-Val-Cit-PABC, MC-Val-Cit-PABC-DMEA, GGFG (Glycine-Glycine-Phenylalanine-Glycine), MC-GGFG-aminomethyl, AcBut (4-(4-acetylphenoxy)-butanoic acid), and AcBut-dimethylhydrazide, preferably MC-Val-Cit-PABC.

28. A pharmaceutical composition comprising the antibody or the antigen-binding fragment thereof according to any one of claims 1-17, or the bi-specific antibody according to claim 18, or the nucleic acid according to claim 19, or the vector according to claim 20, or the host cell according to claim 21, or the antibody-drug conjugate according to any one of claims 22-27, and optionally a pharmaceutically acceptable carrier or excipient.

29. The pharmaceutical composition according to claim 28, wherein the composition further comprises a second therapeutic agent, optionally the second therapeutic agent is selected from the group consisting of an antibody, a chemotherapeutic agent, an siRNA, antisense oligonucleotide, a polypeptide, and a small molecule drug.

30. A method of treating a cancer in a subject, comprising administering to the subject an effective amount of the antibody or the antigen-binding fragment thereof according to any one of claims 1-17, or the bi-specific antibody according to claim 18, or the nucleic acid according to claim 19, or the vector according to claim 20, or the host cell according to claim 21, the antibody-drug conjugate according to any one of claims 22-27, or the pharmaceutical composition according to claim 28 or 29.

31. The method according to claim 30, wherein the cancer is a cancer associated with the expression of CEACAM5 and/or CEACAM6.

32. The method according to claim 31, wherein the cancer is selected from the group consisting of small intestine cancer, colorectal cancer, stomach cancer, lung cancer, cervical cancer, pancreatic cancer, esophagus cancer, ovarian cancer, thyroid cancer, bladder cancer, endometrial cancer, breast cancer, liver cancer, prostatic cancer and skin cancer.

33. The method according to any one of claims 30-32, further comprising administering to the subject a second therapeutic agent.

34. The method according to claim 33, wherein the second therapeutic agent is selected from an antibody, a chemotherapeutic agent, an siRNA, an antisense oligonucleotide, a polypeptide, and a small molecule drug.

35. Use of the antibody or the antigen-binding fragment thereof according to any one of claims 1-17, or the bi-specific antibody according to claim 18, or the nucleic acid according to claim 19, or the vector according to claim 20, or the host cell according to claim 21, the antibody-drug conjugate according to any one of claims 22-27, or the pharmaceutical composition according to claim 28 or 29 in the manufacture of a medicament for treating a cancer in a subject.

36. The use according to claim 35, wherein the cancer is a cancer associated with the expression of CEACAM5 and/or CEACAM6, preferably the cancer is selected from the group consisting of small intestine cancer, colorectal cancer, stomach cancer, lung cancer, cervical cancer, pancreatic cancer, esophagus cancer, ovarian cancer, thyroid cancer, bladder cancer, endometrial cancer, breast cancer, liver cancer, prostatic cancer and skin cancer.

37. The use according to claim 35 or 36, wherein the medicament further comprises a second therapeutic agent, optionally the second therapeutic agent is selected from an antibody, a chemotherapeutic agent, an siRNA, an antisense oligonucleotide, a polypeptide, and a small molecule drug.

38. The use according to claim 35 or 36, wherein the medicament is administered in combination with a second therapeutic agent, optionally the second therapeutic agent is selected from an antibody, a chemotherapeutic agent, an siRNA, an antisense oligonucleotide, a polypeptide, and a small molecule drug.

39. The antibody or the antigen-binding fragment thereof according to any one of claims 1-17, or the bi-specific antibody according to claim 18, or the nucleic acid according to claim 19, or the vector according to claim 20, or the host cell according to claim 21, the antibody-drug conjugate according to any one of claims 22-27, or the pharmaceutical composition according to claim 28 or 29 for use in treating a cancer in a subject.

40. The antibody or the antigen-binding fragment thereof, the bi-specific antibody, the nucleic acid, the vector, the host cell, the antibody-drug conjugate, or the pharmaceutical composition for use according to claim 39, wherein the cancer is a cancer associated with the expression of CEACAM5 and/or CEACAM6, preferably the cancer is selected from the group consisting of small intestine cancer, colorectal cancer, stomach cancer, lung cancer, cervical cancer, pancreatic cancer, esophagus cancer, ovarian cancer, thyroid cancer, bladder cancer, endometrial cancer, breast cancer, liver cancer, prostatic cancer and skin cancer.

41. The antibody or the antigen-binding fragment thereof, the bi-specific antibody, the nucleic acid, the vector, the host cell, the antibody-drug conjugate, or the pharmaceutical composition for use according to claim 39 or 40, further comprising administering to the subject a second therapeutic agent, optionally the second therapeutic agent is selected from an antibody, a chemotherapeutic agent, an siRNA, an antisense oligonucleotide, a polypeptide, and a small molecule drug.

42. A method for detecting the presence or level of CEACAM5 and/or CEACAM6 in a sample, comprising:
(a) contacting the sample with the antibody or the antigen binding fragment thereof according to any one of claims 1-10, and
(b) determining the presence or level of CEACAM5 and/or CEACAM6 in the sample by detecting binding of the antibody to the sample.

43. A method for diagnosing a cancer associated with the expression of CEACAM5 and/or CEACAM6 in a subject comprising:
(a) obtaining a biological sample from the subject,
(b) contacting the sample with the antibody or the antigen binding fragment thereof according to any one of claims 1-10, and
(c) detecting binding of the antibody to the sample,
wherein an increase in binding of the antibody or antigen binding fragment thereof to the sample as compared to binding of the antibody or antigen binging fragment thereof to a control sample identifies the subject as having the cancer.

44. A method for imaging a cancer associated with the expression of CEACAM5 and/or CEACAM6 in a subject comprising:
(a) administering the antibody or antigen binding fragment thereof according to any one of claims 1-10 to the subject, wherein the antibody is conjugated to a detectable marker, and
(b) detecting the presence of the marker.

45. The method according to claim 44, wherein:
(a) the detectable marker is ¹¹¹ In, and preferably the detection of the marker is by single-photon emission computed tomography, or
(b) the detectable marker is ⁸⁹Zr, and preferably the detection of the marker is by positron emission tomography.
